(19) United States
(12) Patent Application Publication
(10) Pub. No.: US 2013/0172294 A1
(43) Pub. Date: Jul. 4, 2013

(54) Treatment with VB-201

(71) Applicants: Yael Cohen, Kiryat-Ono (IL); Naamit Sher, Rechovot (IL); Itzhak Mendel, Rechovot (IL); Niva Yacov, Tel-Aviv (IL); Eyal Breitbart, Hashmonaim (IL)

(72) Inventors: Yael Cohen, Kiryat-Ono (IL); Naamit Sher, Rechovot (IL); Itzhak Mendel, Rechovot (IL); Niva Yacov, Tel-Aviv (IL); Eyal Breitbart, Hashmonaim (IL)

(73) Assignee: Vascular Biogenics Ltd., Or Yehuda (IL)

(21) Appl. No.: 13/520,719

(22) PCT Filed: Jan. 5, 2011
(86) PCT No.: PCT/IL11/00008

§ 371 Date: Mar. 7, 2013

Related U.S. Application Data
(60) Provisional application No. 61/292,226, filed on Jan. 5, 2010.
(60) Provisional application No. 61/282,250, filed on Jan. 7, 2010.
(60) Provisional application No. 61/351,975, filed on Jun. 7, 2010.
(60) Provisional application No. 61/389,318, filed on Oct. 4, 2010.
(60) Provisional application No. 61/423,112, filed on Dec. 15, 2010.

(51) Int. Cl. A61K 31/685

(52) U.S. Cl. CPC A61K 31/685
USPC 514/77; 558/169

(57) ABSTRACT

Unit dosage forms comprising between 1 mg and 100 mg VB-201 and a pharmaceutically acceptable carrier, and formulated for oral administration, are disclosed herein, as well as treatment regimens comprising oral administration of VB-201 once or twice daily for treating an inflammatory disease or disorder.

### FIELD AND BACKGROUND OF THE INVENTION

The present invention, in some embodiments thereof, relates to the field of pharmacology and more particularly, but not exclusively, to a novel dosage form and treatment regimen of the oxidized phospholipid VB-201, which can be efficiently used to treat or prevent inflammation associated diseases and disorders such as, for example, atherosclerosis and related disorders, and autoimmune diseases or disorders such as psoriasis, arthritis, multiple sclerosis and inflammatory bowl disease.

Oxidized phospholipids have been previously described as useful in the treatment of medical conditions such as, for example, cardiovascular diseases, cerebrovascular diseases and inflammatory diseases and disorders.

International Patent Application No. PCT/IL2004/000453 (Publication No. WO 04/106486), by the present assignee, describes oxidized lipids for prevention and treatment of inflammation associated with endogenous oxidized lipids. An exemplary such compound is described and known as CI-201 (1-hexadecyl-2-(4′-carboxybutyl)-glycerol-3-phosphocholine; also referred to in the art as VB-201).

International Patent Application No. PCT/IL01/01080 (Publication No. WO 02/41827), by the present assignee, describes oxidized lipids for prevention and treatment of atherosclerosis and related diseases.

Additional background art includes International Patent Application Nos. PCT/IL05/000735 (Publication No. WO 06/006161), PCT/IL02/00005 (Publication No. WO 02/053092) and PCT/IL08/000,013 (Publication No. WO 08/084,472), all being also by the present assignee.

All of the above cited publications are incorporated by reference as if fully set forth herein.

### SUMMARY OF THE INVENTION

The present inventors and now devised an assay for evaluating the pharmacokinetic parameters of 1-hexadecyl-2-(4′-carboxybutyl)-glycerol-3-phosphocholine (VB-201), so as to determine the dosage and regimen required to beneficially treat inflammation.

The present inventors have thus uncovered that the elimination half-life of VB-201 is in a range of about 27 to 51 hours (i.e., longer than one day), and that plasma concentrations of VB-201 are relatively stable when VB-201 is administered once per day. The present inventors have further uncovered that daily dosages of 80 mg VB-201 per day or less are safe and well-tolerated, that a substantial percentage of VB-201 is absorbed into the bloodstream, and that absorption is not strongly affected by whether the subject has recently eaten. The present inventors have further uncovered that daily dosages of 80 mg VB-201 and less are effective at inhibiting inflammatory processes.

According to an aspect of some embodiments of the invention, there is provided a pharmaceutical composition unit dosage form comprising between 1 mg and 100 mg VB-201 and a pharmaceutically acceptable carrier, the pharmaceutical composition unit dosage form being formulated for oral administration.

According to an aspect of some embodiments of the invention, there is provided a use of VB-201 in the manufacture of a unit dosage form of a medicament for treating or preventing an inflammatory disease or disorder, the unit dosage form comprising from 1 mg to 100 mg VB-201 and being formulated for oral administration.

According to an aspect of some embodiments of the invention, there is provided a method of treating or preventing an inflammatory disease or disorder, the method comprising orally administering to a subject in need thereof a therapeutically effective amount of VB-201, wherein the therapeutically effective amount ranges from 1 mg per day to 100 mg per day.

According to an aspect of some embodiments of the invention, there is provided a method of treating or preventing psoriasis, the method comprising orally administering to a subject in need thereof a therapeutically effective amount of VB-201, wherein the therapeutically effective amount ranges from 1 mg per day to 100 mg per day.

According to an aspect of some embodiments of the invention, there is provided a method of treating or preventing rheumatoid arthritis, the method comprising orally administering to a subject in need thereof a therapeutically effective amount of VB-201, wherein the therapeutically effective amount ranges from 1 mg per day to 100 mg per day.

According to an aspect of some embodiments of the invention, there is provided a method of treating or preventing an inflammation selected from the group consisting of inflammation of a carotid artery and inflammation of an aorta, the method comprising orally administering to a subject in need thereof a therapeutically effective amount of VB-201, wherein the therapeutically effective amount ranges from 1 mg per day to 100 mg per day.

According to an aspect of some embodiments of the invention, there is provided a method of treating or preventing an inflammatory disease or disorder, the method comprising orally administering to a subject in need thereof a therapeutically effective amount of VB-201, wherein the administering is effected once or twice a day.

According to an aspect of some embodiments of the invention, there is provided a method of treating or preventing psoriasis, the method comprising orally administering to a subject in need thereof a therapeutically effective amount of VB-201, wherein the administering is effected once or twice a day.

According to an aspect of some embodiments of the invention, there is provided a method of treating or preventing rheumatoid arthritis, the method comprising orally administering to a subject in need thereof a therapeutically effective amount of VB-201, wherein the administering is effected once or twice a day.

According to an aspect of some embodiments of the invention, there is provided a method of treating or preventing an inflammation selected from the group consisting of inflammation of a carotid artery and inflammation of an aorta, the method comprising orally administering to a subject in need thereof a therapeutically effective amount of VB-201, wherein the administering is effected once or twice a day.

According to some embodiments of the invention, the unit dosage form comprises 10 mg VB-201.

According to some embodiments of the invention, the unit dosage form comprises 20 mg VB-201.

According to some embodiments of the invention, the unit dosage form comprises 30 mg VB-201.

According to some embodiments of the invention, the unit dosage form comprises 40 mg VB-201.

According to some embodiments of the invention, the unit dosage form comprises 50 mg VB-201.

According to some embodiments of the invention, the unit dosage form comprises 60 mg VB-201.

According to some embodiments of the invention, the unit dosage form comprises 70 mg VB-201.

According to some embodiments of the invention, the unit dosage form comprises 80 mg VB-201.

According to some embodiments of the invention, the unit dosage form comprises 90 mg VB-201.

According to some embodiments of the invention, the unit dosage form comprises 100 mg VB-201.

According to some embodiments of the invention, the unit dosage form comprises from 10 mg to 30 mg VB-201.

According to some embodiments of the invention, the unit dosage form comprises from 15 mg to 25 mg VB-201.

According to some embodiments of the invention, the unit dosage form comprises from 18 mg to 22 mg VB-201.

According to some embodiments of the invention, the unit dosage form comprises from 70 mg to 90 mg VB-201.

According to some embodiments of the invention, the unit dosage form comprises from 75 mg to 85 mg VB-201.

According to some embodiments of the invention, the pharmaceutical composition unit dosage form is packaged in a packaging material and identified in print, in or on the packaging material, for use in the treatment or prevention of an inflammatory disease or disorder.

According to some embodiments of the invention, the pharmaceutical composition unit dosage form is identified for use once per day.

According to some embodiments of the invention, the pharmaceutical composition unit dosage form is identified for use such that a daily dose consists of one or two units of the pharmaceutical composition unit dosage form.

According to some embodiments of the invention, the pharmaceutical composition unit dosage form is identified for use such that a daily dose consists of one unit of the pharmaceutical composition unit dosage form.

According to some embodiments of the invention, the pharmaceutical composition unit dosage form is identified for use during, or after a meal.

According to some embodiments of the invention, the therapeutically effective amount of VB-201 is administered once per day.

According to some embodiments of the invention, the therapeutically effective amount ranges from 1 mg per day to 100 mg per day.

According to some embodiments of the invention, the therapeutically effective amount is 10 mg VB-201 per day.

According to some embodiments of the invention, the therapeutically effective amount is 20 mg VB-201 per day.

According to some embodiments of the invention, the therapeutically effective amount is 30 mg VB-201 per day.

According to some embodiments of the invention, the therapeutically effective amount is 40 mg VB-201 per day.

According to some embodiments of the invention, the therapeutically effective amount is 50 mg VB-201 per day.

According to some embodiments of the invention, the therapeutically effective amount is 60 mg VB-201 per day.

According to some embodiments of the invention, the therapeutically effective amount is 70 mg VB-201 per day.

According to some embodiments of the invention, the therapeutically effective amount is 80 mg VB-201 per day.

According to some embodiments of the invention, the therapeutically effective amount is 90 mg VB-201 per day.

According to some embodiments of the invention, the therapeutically effective amount is 100 mg VB-201 per day.

According to some embodiments of the invention, the therapeutically effective amount is in a range of from 10 mg to 30 mg VB-201 per day.

According to some embodiments of the invention, the therapeutically effective amount is in a range of from 15 mg to 25 mg VB-201 per day.

According to some embodiments of the invention, the therapeutically effective amount is in a range of from 18 mg to 22 mg VB-201 per day.

According to some embodiments of the invention, the therapeutically effective amount is in a range of from 70 mg to 90 mg VB-201 per day.

According to some embodiments of the invention, the therapeutically effective amount is in a range of from 75 mg to 85 mg VB-201 per day.

According to some embodiments of the invention, a method described herein comprises administering one or two units of a pharmaceutical composition unit dosage form described herein.

According to some embodiments, a method described herein comprises administering one unit of a pharmaceutical composition unit dosage form described herein.

According to some embodiments, the therapeutically effective amount of VB-201 is administered during, or after a meal.

According to some embodiments of the invention, the inflammatory disease or disorder is associated with an endogenous oxidized lipid.

According to some embodiments of the invention, the inflammatory disease or disorder is selected from the group consisting of an idiopathic inflammatory disease or disorder, a chronic inflammatory disease or disorder, an acute inflammatory disease or disorder, an autoimmune disease or disorder, an infectious disease or disorder, an inflammatory malignant disease or disorder, an inflammatory transplantation-related disease or disorder, an inflammatory degenerative disease or disorder, a disease or disorder associated with a hypersensitivity, an inflammatory cardiovascular disease or disorder, an inflammatory cerebrovascular disease or disorder, a peripheral vascular disease or disorder, an inflammatory glandular disease or disorder, an inflammatory gastrointestinal disease or disorder, an inflammatory cutaneous disease or disorder, an inflammatory hepatic disease or disorder, an inflammatory neurological disease or disorder, an inflammatory musculo-skeletal disease or disorder, an inflammatory renal disease or disorder, an inflammatory reproductive disease or disorder, an inflammatory systemic disease or disorder, an inflammatory connective tissue disease or disorder, an inflammatory tumor, necrosis, an inflammatory implant-related disease or disorder, an inflammatory aging process, an immunodeficiency disease or disorder, a proliferative disease or disorder and an inflammatory pulmonary disease or disorder.

According to some embodiments of the invention, the inflammatory disease or disorder is selected from the group consisting of psoriasis, rheumatoid arthritis, multiple sclerosis, inflammatory bowel disease, atherosclerosis, inflammation of a carotid artery and inflammation of an aorta.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a graph showing mean VB-201 plasma concentrations as a function of time following a single-dose oral administration of 1 mg (Cohort 1), 3 mg (Cohort 2), 10 mg (Cohort 3), 30 mg (Cohort 4), and 50 mg (Cohort 7) VB-201 capsules;

FIG. 2 is a graph showing mean VB-201 plasma concentrations as a function of time on day 1 and day 14, following a daily oral administration of 5 mg (Chrt 1), 10 mg (Chrt 2), 20 mg (Chrt 3b) and 30 mg (Chrt 4) VB-201;

FIG. 3 is a graph showing mean VB-201 plasma concentrations as a function of time following a single-dose administration of one (1×10) or two (10+10) 10 mg capsules, for either enteric coated capsules (Coat) and uncoated capsules (UnCoat), and either to fasting subjects (Fast) or to fed subjects (Fed);

FIG. 4 is a graph showing mean VB-201 plasma concentrations as a function of time on day 1 and day 14, following a daily oral administration of 40 mg VB-201 (Cohort 1), and on day 14, following a daily oral administration of 80 mg VB-201 (Cohort 2);

FIGS. 5A-5C are graphs showing mean C-reactive protein (CRP) plasma levels before (day 0) and after (day 14 or day 28) daily oral administration of 40 mg VB-201 for 14 days (FIG. 5A), 80 mg VB-201 for 14 days (FIG. 5B) or 80 mg VB-201 for 28 days (FIG. 5C), as determined by high sensitivity CRP (hsCRP) assay (N indicates number of subjects tested, and % reduction in CRP levels are shown);

FIGS. 6A-6D are graphs showing interleukin-23 (IL-23) plasma levels (FIGS. 6A and 6C) and interferon-γ (IFN-γ) levels (FIGS. 6B and 6D) in subjects who had pre-dosing (day 0) detectable levels of IL-23 and were administered 40 mg VB-201 per day for 14 days (FIGS. 6A-6B) and 80 mg VB-201 per day for 28 days (FIGS. 6C-6D); and

FIG. 7 is a graph showing the degree of migration (expressed as the number of migrated cells), over the course of 3.5 hours, of human CD14 cells (pre-treated for 30 minutes with 8 μM VB-201 or with control solvent) towards RANTES (100 ng/ml), MIP-1α (50 ng/ml), MCP-1 (50 ng/ml) or MCP-3 (50 ng/ml).

### DESCRIPTION OF SPECIFIC EMBODIMENTS OF THE INVENTION

The present invention, in some embodiments thereof, relates to a novel dosage form and treatment regimen of the oxidized phospholipid VB-201, which can be efficiently used to treat or prevent inflammatory diseases and disorders such as, for example, atherosclerosis and related disorders, inflammatory bowel diseases and autoimmune diseases or disorders such as psoriasis and arthritis.

The principles and operation of the present invention may be better understood with reference to the figures and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

VB-201 (also referred to herein and in the art as CI-201) has shown considerable promise as a therapeutically active agent in various in vitro models and in vivo animal models of inflammatory conditions.

In an attempt to improve treatment of inflammatory diseases and disorders, the present inventors have studied in detail the safety, tolerability and pharmacokinetics of VB-201 in human subjects. To this effect, the present inventors have put ample efforts in devising pharmacological assays for determining the safety, tolerability and pharmacokinetics of VB-201 in human subjects. The protocols of these assays are described in detail in the Examples section that follows. Based on the data obtained in the clinical studies conducted, the present inventors have developed optimal treatment regimens and unit dosage forms.

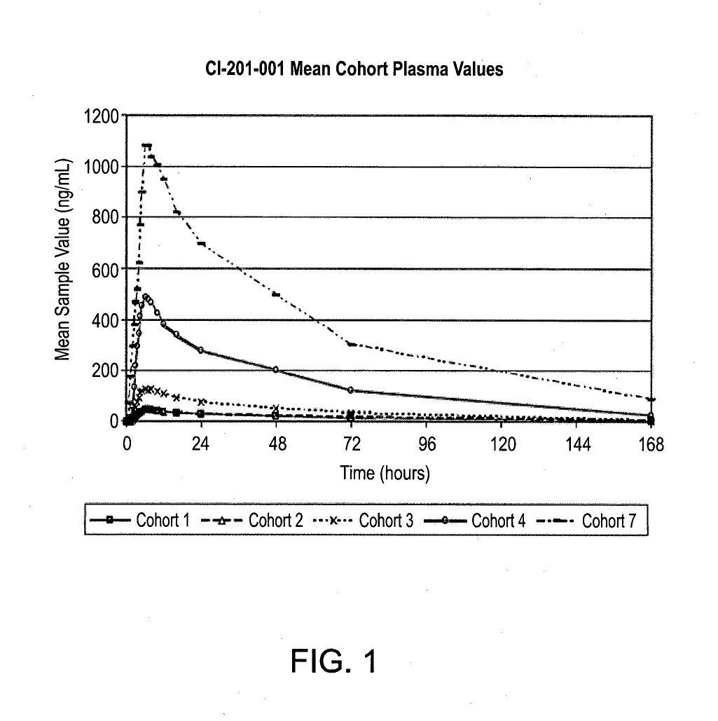

Referring now to the drawings and tables, FIG. 1 shows the time profile of plasma concentrations of VB-201 (also referred to herein as a “concentration-time curve”) following oral administration of 1 to 50 mg VB-201. Table 1 presented in the Examples section provides values of various pharmacokinetic parameters derived from concentration-time curves. Table 1 shows, for example, that a substantial percentage of VB-201 is absorbed into the bloodstream

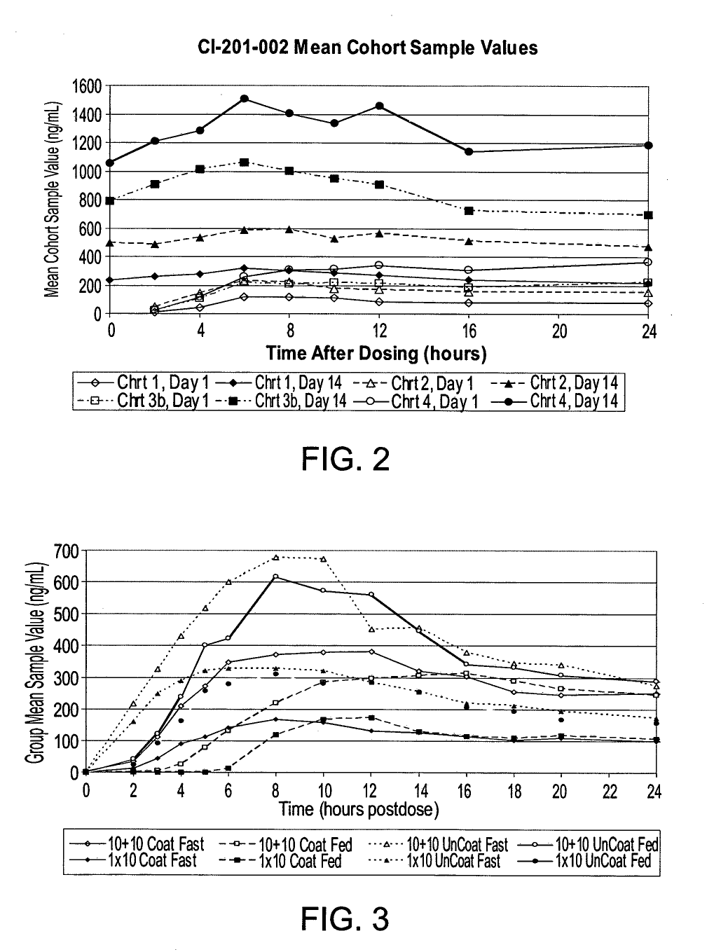

FIG. 2 shows concentration-time curves on day 1 and on day 14 of a regimen of daily oral administration of 5-30 mg VB-201. The accumulation of VB-201 in the blood is clearly shown therein. Table 2 presented in the Examples section provides values of various pharmacokinetic parameters for both day 1 and day 14.

FIG. 3 shows that plasma concentration levels of VB-201 were not significantly affected by whether the subject was administered VB-201 when fed or when fasting.

FIG. 3 further shows that enteric coatings on VB-201 capsules reduced the amount of VB-201 which was absorbed, and that enteric coatings delayed absorption of VB-201, particularly when the subject was fed prior to administration. Table 3 presented in the Examples section provides values of various pharmacokinetic parameters for both fed and fasting subjects, and for both coated and non-coated capsules.

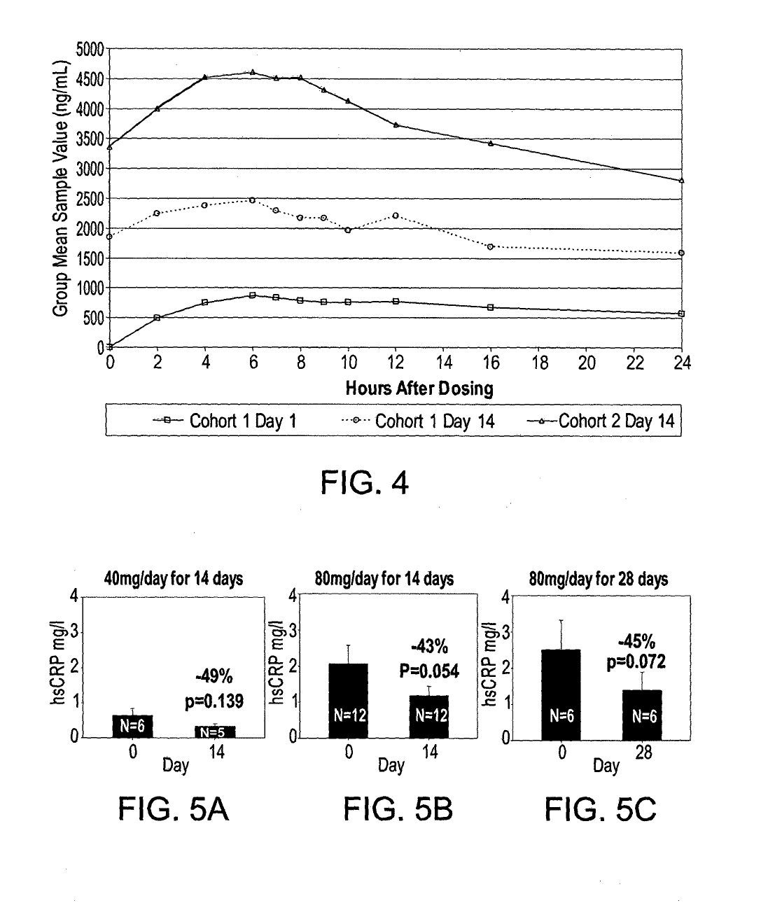

FIG. 4 shows concentration-time curves on day 14 of a regimen of daily oral administration of 40 or 80 mg VB-201. The concentration-time curve on day 1 is shown for 40 mg per day VB-201, such that the accumulation of VB-201 in the blood is clearly shown therein. Table 4 presented in the Examples section provides values of various pharmacokinetic parameters derived from the data for day 1 and day 14.

The abovementioned FIGS. 1-4 and Tables 1-4 further show that plasma concentrations of VB-201 are relatively linearly proportional to the administered dose of VB-201.

FIGS. 2 and 4 further show that plasma concentrations of VB-201 are relatively stable during over the course of a day when VB-201 is regularly administered once-daily. Thus, minimum concentrations during a 24-hour period were at least about 66.7% of the maximum concentrations during the same time period.

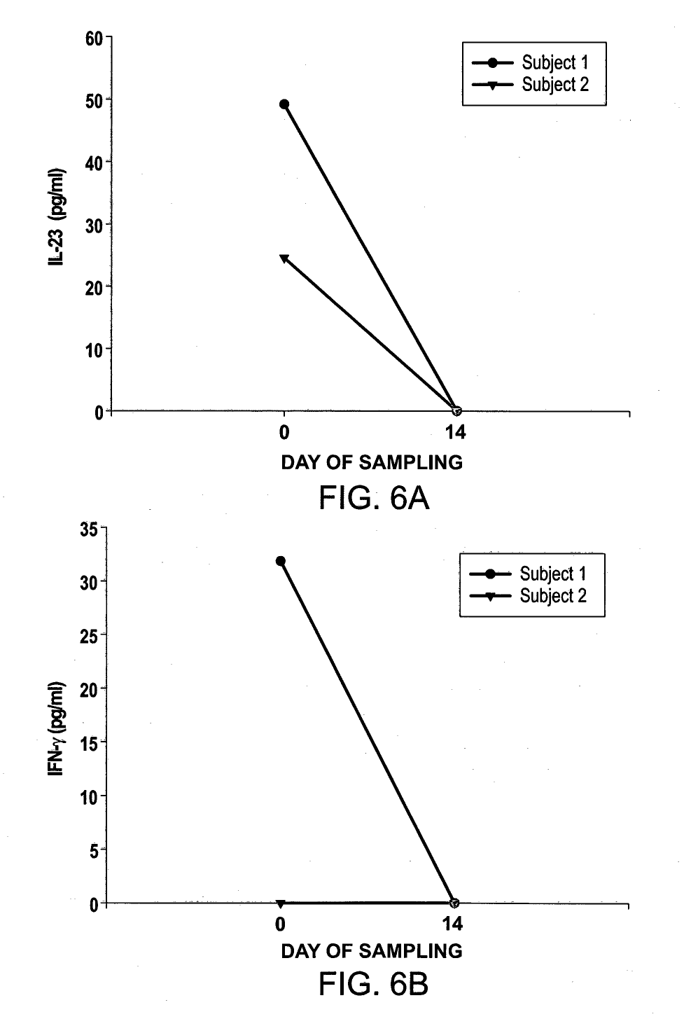

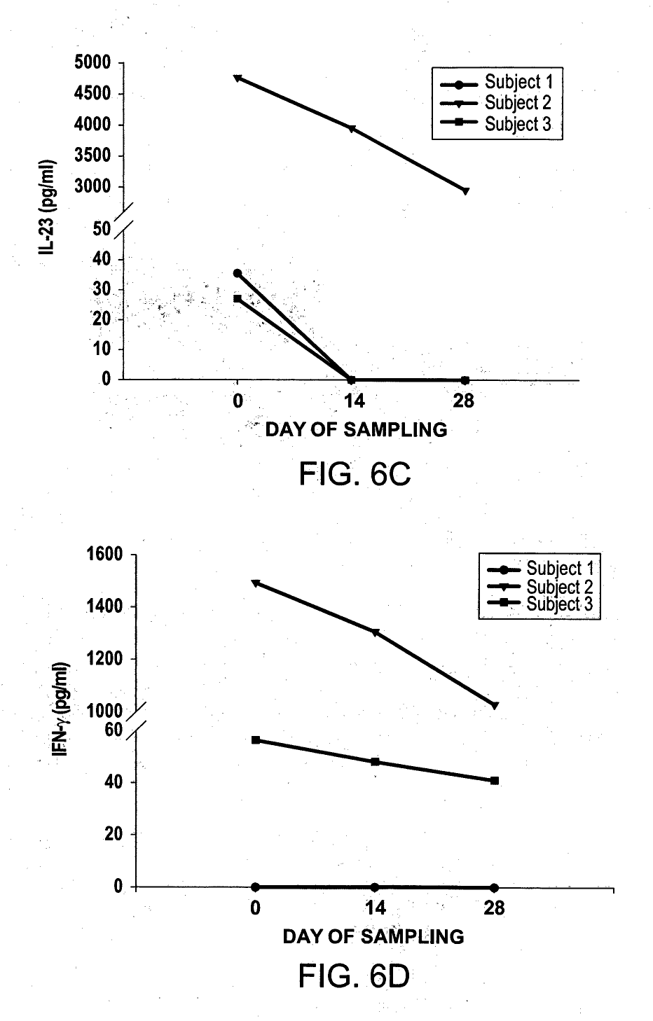

FIGS. 5A-5C show that administration of 40 mg per day and 80 mg per day VB-201 considerable reduces C-reactive protein levels, a marker of inflammation. FIGS. 6A-6D show that administration of 40 or 80 mg per day VB-201 reduces levels of the pro-inflammatory cytokines interleukin-23 and interferon-γ. These results indicate that the dosages described herein are effective at inhibiting inflammatory processes.

As demonstrated in the experimental results presented in the Examples section below, repeated oral administration of VB-201 at a daily dose of 80 mg was well-tolerated by subjects, and no substantial toxicity was observed.

Hence, according to an aspect of some embodiments of the invention there is provided a method of treating or preventing an inflammatory disease or disorder, the method comprising orally administering to a subject in need thereof a therapeutically effective amount of VB-201. The therapeutically effective amount ranges from 1 mg per day to 100 mg per day. Optionally, the therapeutically effective amount ranges from 10 mg per day to 100 mg per day.

The therapeutically effective amounts described herein refer to therapeutically effective amounts for administration to adult subjects. Optionally, amounts of VB-201 to be administered a child are adjusted, for example, according to body weight of the child.

According to some embodiments, the therapeutically effective amounts described herein are absolute, and do not depend on the body weight of the subject, with the optional exception of subjects with a body weight far from the adult average (e.g., children).

According to alternative embodiments, the therapeutically effective amounts described herein are considered suitable for a subject of average body weight (e.g., 70 kg), and the amount to be administered is adjusted according to the body weight of the subject receiving VB-201. For example, a dose of 100 mg per day recited herein is optionally understood to refer to a dose of 100 mg per day per 70 kg body weight (i.e., 1.43 mg/kg per day).

As further demonstrated in the experimental results presented in the Examples section below, the elimination half-life (T_{1/2}) of VB-201 is in a range of about 27 to 51 hours (i.e., longer than one day). In addition, the VB-201 enters the blood stream gradually following administration, with peak plasma concentrations usually occurring approximately 6-11 hours after administration. As a result of the aforementioned kinetics of VB-201 plasma concentrations, the plasma concentrations of VB-201 are relatively stable when VB-201 is administered once per day, and are expected to be even more stable when VB-201 is administered twice per day.

Hence, according to an aspect of some embodiments of the invention there is provided a method of treating or preventing an inflammatory disease or disorder, the method comprising orally administering to a subject in need thereof a therapeutically effective amount of VB-201, wherein administration of the VB-201 is effected once or twice a day.

According to exemplary embodiments, the administration of VB-201 is effected once a day.

Regimens including administration of a therapeutically effective agent once or twice a day (and particularly for administration once a day) are particularly convenient and useful, when the pharmacokinetics of the agent is suitable for such a regimen, as the administration is readily incorporated in a daily routine. Consequently, forgetting to administer the agent is less likely to occur. Moreover, once or twice daily administration of an agent is not particularly burdensome on either the subject or any other person (e.g., medical professional administering) administering the agent. Consequently, refusal to accept and/or provide administration is less likely to occur. As a result of the aforementioned factors, a regimen of once or twice daily administration is likely to induce high compliance to the regimen. A regimen of once daily administration is particularly likely to induce high compliance.

According to some embodiments, the therapeutically effective amount of VB-201 is in a range of 1 mg per day to 100 mg per day, optionally in a range of 10 mg per day to 100 mg per day, and administering this amount is effected once or twice per day, depending on the unit dosage form utilized, as detailed hereinafter, and is optionally effected once per day.

In some embodiments, the therapeutically effective amount is 10 mg per day, in some embodiments it is 20 mg per day, in some embodiments it is 30 mg per day, in some embodiments it is 40 mg per day, in some embodiments it is 50 mg per day, in some embodiments it is 60 mg per day, in some embodiments it is 70 mg per day, in some embodiments it is 80 mg per day, in some embodiments it is 90 mg per day, and in some embodiments it is 100 mg per day. Any integer between 1 to 100 mg per day is also contemplated.

According to some exemplary embodiments, the therapeutically effective amount is approximately 20 mg per day (e.g., in a range of from 10 mg per day to 30 mg per day). Optionally, the amount is in a range of from 15 mg per day to 25 mg per day, and optionally in a range of from 18 mg per day to 22 mg per day.

According to other exemplary embodiments, the therapeutically effective amount is approximately 80 mg per day (e.g., in a range of from 70 mg per day to 90 mg per day). Optionally, the amount is in a range of from 75 mg per day to 85 mg per day, and optionally in a range of from 88 mg per day to 92 mg per day.

According to some embodiments, the therapeutically effective amount of VB-201 is administered during a meal or after a meal.

As used herein, the term “during” refers to a period of time from 60 minutes before the beginning of the meal, optionally 30 minutes before the meal, optionally 10 minutes before the meal, and optionally 5 minutes before the meal, until 60 minutes after the meal ends, optionally 30 minutes after the meal, optionally 10 minutes after the meal, and optionally 5 minutes after the meal.

According to some embodiments, the phrase “after a meal” refers to a period of time lasting until 6 hours after the end of the meal, optionally, 4 hours after the meal, optionally 3 hours after the meal, and optionally 2 hours after the meal.

Alternatively or additionally, “after a meal” encompasses a period of time during which the subject feels a sensation of a “full stomach” rather than an “empty stomach”.

Without being bound by any particular theory, it is believed that administration during or after a meal reduces the chances of experiencing possible side effects of VB-201.

As exemplified in the Examples section that follows, VB-201 may be administered within a coating (e.g., an enteric coating). Exemplary materials for forming a coating include Eudragit®, Opadry® (e.g., Opadry® amb) and Acryl-Eze®, which may be used alone or in combination to form an enteric coating.

The coating may be used, for example, for protecting a portion of the digestive system (e.g., the stomach) from the VB-201 (e.g., in order to minimize side effects) or any substance (e.g., carrier) co-administered with the VB-201, to mask a taste of VB-201 or any substance (e.g., carrier) co-administered with the VB-201 (e.g., to enhance palatability), and/or to control release and/or uptake (e.g., delaying release and/or uptake) of VB-201 (e.g., in order to provide for more stable plasma concentrations of VB-201).

As demonstrated in the Examples section, an enteric coating delays uptake of VB-201 most significantly when the VB-201 is administered to a fed subject. Consequently, in embodiments wherein a coating (e.g., an enteric coating) is intended to delay uptake, the VB-201 is optionally administered during or after a meal.

In embodiments where VB-201 is administered without an enteric coating, VB-201 can be administered either to a fasting subject or to a fed subject.

The relatively narrow range of doses found to be suitable according to embodiments of the present invention allows for the production of unit dosage forms of a medicament (e.g., a pharmaceutical composition) designed for easy and convenient administration of a therapeutically effective amount of VB-201 described herein. It may be beneficial for the therapeutically effective amount to consist of no more than two unit dosage forms, as administration of more than two dosage forms at any one time may be more difficult. For example, it is commonly difficult to swallow more than two solid unit dosage forms (e.g., pills, tablets, capsules, etc.). Moreover, administration of more than two unit dosage forms at any one time increases the likelihood of confusion and administration of an inappropriate dose.

Hence, according to optional embodiments, the method comprises administering one or two units of a unit dosage form at each administration. Optionally, one unit of the unit dosage form is administered per administration.

According to some embodiments, the method is effected by administering one or two units (optionally one unit) of a unit dosage form described herein.

In view of the abovementioned benefits of suitable unit dosage forms, according to another aspect of embodiments of the present invention, there is provided a use of VB-201 in the manufacture of a unit dosage form of a medicament (e.g., a pharmaceutical composition) for treating or preventing an inflammatory disease or disorder, the unit dosage form comprising from 1 mg to 100 mg VB-201, and being formulated for oral administration. Optionally, the unit dosage form comprises from 10 mg to 100 mg VB-201.

In any of the methods and uses described herein, the VB-201 can be utilized either per se or as a part of a pharmaceutical composition, which further comprises a pharmaceutically acceptable carrier.

Hence, according to another aspect of embodiments of the present invention, there is provided a pharmaceutical composition unit dosage form comprising between 1 mg and 100 mg of VB-201 and a pharmaceutically acceptable carrier. The pharmaceutical composition unit dosage form is formulated for oral administration. Optionally, the pharmaceutical composition unit dosage form comprises between 10 mg and 100 mg of VB-201.

According to optional embodiments of the present invention, such a pharmaceutical composition unit dosage form is packaged in a packaging material and identified in print, in or on the packaging material, for use in the treatment or prevention of an inflammatory disease or disorder. In some embodiments, a plurality of unit dosage forms is packaged in a packaging material and identified in print, in or on the packaging material, for use in the treatment or prevention of an inflammatory disease or disorder. Suitable inflammatory diseases and disorders are discussed in more detail elsewhere herein.

As used herein, a “pharmaceutical composition” refers to a preparation of VB-201 (as active ingredient), or physiologically acceptable salts or prodrugs thereof, with other chemical components, including, but not limited to, physiologically suitable carriers, excipients, lubricants, buffering agents, antibacterial agents, bulking agents (e.g. mannitol), antioxidants (e.g., ascorbic acid or sodium bisulfite), and the like. The purpose of the pharmaceutical composition is to facilitate administration of VB-201 to a subject.

The term “unit dosage form”, as used herein, describes physically discrete units, each unit containing a predetermined quantity of VB-201 calculated to produce the desired therapeutic effect, in association with at least one pharmaceutically acceptable carrier, diluent, excipient, or combination thereof.

Herein, the phrases “physiologically acceptable carrier” and “pharmaceutically acceptable carrier”, which are used interchangeably, describe a carrier or a diluent that does not cause significant irritation to the subject and does not abrogate the biological activity and properties of the VB-201.

As used herein, the term “carrier” refers to a diluent, adjuvant, excipient, or vehicle with which the therapeutic is administered.

Herein the term “excipient” refers to an inert substance added to a pharmaceutical composition to further facilitate administration of an active ingredient.

Techniques for formulation and administration of drugs may be found in “Remington's Pharmaceutical Sciences” Mack Publishing Co., Easton, Pa., latest edition, which is incorporated herein by reference.

Pharmaceutical compositions for use in accordance with the present invention thus may be formulated for oral administration in a conventional manner using one or more pharmaceutically acceptable carriers comprising excipients and auxiliaries, which facilitate processing of the compounds into preparations which can be used pharmaceutically.

A pharmaceutical composition unit dosage form according to some embodiments can be formulated readily by combining VB-201 with pharmaceutically acceptable carriers well known in the art. Using such carriers the active ingredient (VB-201) is formulated, for example, as sachets, pills, caplets, capsules, tablets, dragee-cores or discrete (e.g., separately packaged) units of powder, granules, or suspensions or solutions in water or non-aqueous media. Thickeners, diluents, flavorings, dispersing aids, emulsifiers or binders may be desirable.

Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredient may be dissolved or suspended in suitable liquids, such as fatty oils, liquid paraffin, or liquid polyethylene glycols. In addition, stabilizers may be added. Preferably, formulations for oral administration further include a protective coating, aimed at protecting or slowing enzymatic degradation of the preparation in the GI tract.

Composition unit dosage forms according to the present embodiments may, if desired, be presented in a pack or dispenser device, such as an FDA (the U.S. Food and Drug Administration) approved kit, which may contain one or more unit dosage forms containing VB-201. The pack or dispenser device may, for example, comprise metal or plastic foil, such as, but not limited to a blister pack. The pack or dispenser device may be accompanied by instructions for administration. The pack may also be accompanied by a notice associated with the container in a form prescribed by a governmental agency regulating the manufacture, use or sale of pharmaceuticals, which notice is reflective of approval by the agency of the form of the compositions for human administration. Such notice, for example, may be of labeling approved by the U.S. Food and Drug Administration for prescription drugs or of an approved product insert. Composition unit dosage forms comprising VB-201 formulated in a compatible pharmaceutical carrier may also be prepared, placed in an appropriate container, and labeled for treatment of an inflammatory disease or disorder, as defined herein.

According to some embodiments, the unit dosage for is identified (e.g., in the abovementioned instructions for administration) for use once or twice per day, optionally once per day.

According to some embodiments, the unit dosage form is identified (e.g., in the abovementioned instructions for administration) for use such that a daily dose consists of one or two units of the unit dosage form, and optionally one unit of the unit dosage form.

According to some embodiments, the unit dosage form is identified (e.g., in the abovementioned instructions for administration) for use during, or after a meal, as described herein.

In some embodiments, the unit dosage form comprises 10 mg VB-201. Such a unit dosage form is particularly suitable for administering 10 mg VB-201 per day or 20 mg VB-201 per day (e.g., by administering one unit dosage form twice per day or administering 2 unit dosage forms once a day). Such unit dosage forms are also relatively suitable for administering 30 mg or 40 mg VB-201 per day (e.g., by administering 3 or 4 unit dosage forms per day, optionally by administering 1 or 2 unit dosage forms twice a day).

In some embodiments, the unit dosage form comprises 20 mg VB-201. Such a unit dosage form is particularly suitable for administering 20 mg VB-201 per day or 40 mg VB-201 per day (e.g., by administering one unit dosage form twice per day or administering 2 unit dosage forms once a day). Such unit dosage forms are also relatively suitable for administering 60 mg or 80 mg VB-201 per day (e.g., by administering 3 or 4 unit dosage forms per day, optionally by administering 1 or 2 unit dosage forms twice a day).

In some embodiments, the unit dosage form comprises 30 mg VB-201. Such a unit dosage form is particularly suitable for administering 30 mg VB-201 per day or 60 mg VB-201 per day (e.g., by administering one unit dosage form twice per day or administering 2 unit dosage forms once a day). Such unit dosage forms are also relatively suitable for administering 90 mg VB-201 per day (e.g., by administering 3 unit dosage forms per day, optionally by administering 1 or 2 unit dosage forms twice a day).

In some embodiments, the unit dosage form comprises 40 mg VB-201. Such a unit dosage form is particularly suitable for administering 40 mg VB-201 per day or 80 mg VB-201 per day (e.g., by administering one unit dosage form twice per day or administering 2 unit dosage forms once a day).

In some embodiments, the unit dosage form comprises 50 mg VB-201. Such a unit dosage form is particularly suitable for administering 50 mg VB-201 per day or 100 mg VB-201 per day (e.g., by administering one unit dosage form twice per day or administering 2 unit dosage forms once a day).

In some embodiments, the unit dosage form comprises 60 mg VB-201. Such a unit dosage form is particularly suitable for administering 60 mg VB-201 per day.

In some embodiments, the unit dosage form comprises 70 mg VB-201, in some embodiments it comprises 80 mg VB-201, in some embodiments it comprises 90 mg VB-201, and in some embodiments it comprises 100 mg VB-201. Such unit dosage forms are particularly suitable for administering 70 mg per day, 80 mg per day, 90 mg per day, and 100 mg per day, respectively, of VB-201.

In some embodiments, the unit dosage form comprises 80 mg VB-201. Such a unit dosage form is particularly suitable for administering 80 mg VB-201 per day, once a day.

According to some exemplary embodiments, the unit dosage form comprises approximately 20 mg VB-201 (e.g., in a range of from 10 mg to 30 mg). Optionally, the amount of VB-201 in the unit dosage form is in a range of from 15 mg to 25 mg, and optionally in a range of from 18 mg to 22 mg.

According to other exemplary embodiments, the therapeutically effective amount is approximately 80 mg (e.g., in a range of from 70 mg to 90 mg). Optionally, the amount of VB-201 in the unit dosage form is in a range of from 75 mg to 85 mg, and optionally in a range of from 88 mg to 92 mg.

Optionally, a unit dosage form is designed so as to facilitate division of a unit dosage form into two half-unit dosage forms. For example, a pill, tablet or caplet may be scored so as to be readily broken in half.

VB-201 (1-hexadecyl-2-(4′-carboxybutyl)-glycerol-3-phosphocholine) according to embodiments of the present invention may be a chiral enantiomer of 1-hexadecyl-2-(4′-carboxybutyl)-glycerol-3-phosphocholine, i.e., either the (R)-enantiomer ((R)-1-hexadecyl-2-(4′-carboxybutyl)-sn-glycerol-3-phosphocholine) or the (S)-enantiomer ((S)-1-hexadecyl-2-(4′-carboxybutyl)-glycerol-3-phosphocholine), or a mixture thereof (e.g., a racemate). According to exemplary embodiments, VB-201 is (R)-1-hexadecyl-2-(4′-carboxybutyl)-sn-glycerol-3-phosphocholine.

According to optional embodiments of the methods, uses and pharmaceutical composition unit dosage forms described herein, the inflammatory disease or disorder treatable according to embodiments of the present invention is an inflammatory disease or disorder associated with an endogenous oxidized lipid.

As used herein, the phrase “an endogenous oxidized lipid” refers to one or more oxidized lipids that are present or formed in vivo, as a result of inflammatory and other cell- or humoral-mediated processes. Oxidized low-density lipoprotein (oxidized-LDL) is an example of an endogenous oxidized lipid associated with an inflammatory disease or disorder.

Inflammatory diseases or disorders treatable according to exemplary embodiments of the present invention include psoriasis (e.g., plaque psoriasis), rheumatoid arthritis, and atherosclerosis and related conditions, such as inflammation of an artery (e.g., inflammation of a carotid artery and/or inflammation of an aorta).

Additional examples of inflammatory diseases or disorders treatable according to exemplary embodiments of the present invention include multiple sclerosis and inflammatory bowel disease (e.g., chronic inflammatory bowel disease).

Representative inflammatory diseases and disorders treatable according to embodiments of the present invention include, for example, idiopathic inflammatory diseases or disorders, chronic inflammatory diseases or disorders, acute inflammatory diseases or disorders, autoimmune diseases or disorders, infectious diseases or disorders, inflammatory malignant diseases or disorders, inflammatory transplantation-related diseases or disorders, inflammatory degenerative diseases or disorders, diseases or disorders associated with a hypersensitivity, inflammatory cardiovascular diseases or disorders, inflammatory cerebrovascular diseases or disorders, peripheral vascular diseases or disorders, inflammatory glandular diseases or disorders, inflammatory gastrointestinal diseases or disorders, inflammatory cutaneous diseases or disorders, inflammatory hepatic diseases or disorders, inflammatory neurological diseases or disorders, inflammatory musculo-skeletal diseases or disorders, inflammatory renal diseases or disorders, inflammatory reproductive diseases or disorders, inflammatory systemic diseases or disorders, inflammatory connective tissue diseases or disorders, inflammatory tumors, necrosis, inflammatory implant-related diseases or disorders, inflammatory aging processes, immunodeficiency diseases or disorders, proliferative diseases and disorders and inflammatory pulmonary diseases or disorders, as is detailed hereinbelow.

Non-limiting examples of hypersensitivities include Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity, delayed type hypersensitivity, helper T lymphocyte mediated hypersensitivity, cytotoxic T lymphocyte mediated hypersensitivity, TH1 lymphocyte mediated hypersensitivity, and TH2 lymphocyte mediated hypersensitivity.

Non-limiting examples of inflammatory cardiovascular disease or disorder include occlusive diseases or disorders, atherosclerosis, a cardiac valvular disease, stenosis, restenosis, in-stent-stenosis, myocardial infarction, coronary arterial disease, acute coronary syndromes, congestive heart failure, angina pectoris, myocardial ischemia, thrombosis, Wegener's granulomatosis, Takayasu's arteritis, Kawasaki syndrome, anti-factor VIII autoimmune disease or disorder, necrotizing small vessel vasculitis, microscopic polyangiitis, Churg and Strauss syndrome, pauci-immune focal necrotizing glomerulonephritis, crescentic glomerulonephritis, antiphospholipid syndrome, antibody induced heart failure, thrombocytopenic purpura, autoimmune hemolytic anemia, cardiac autoimmunity, Chagas' disease or disorder, and anti-helper T lymphocyte autoimmunity.

Stenosis is an occlusive disease of the vasculature, commonly caused by atheromatous plaque and enhanced platelet activity, most critically affecting the coronary vasculature.

Restenosis is the progressive re-occlusion often following reduction of occlusions in stenotic vasculature. In cases where patency of the vasculature requires the mechanical support of a stent, in-stent-stenosis may occur, re-occluding the treated vessel.

Non-limiting examples of cerebrovascular diseases or disorders include stroke, cerebrovascular inflammation, cerebral hemorrhage and vertebral arterial insufficiency.

Non-limiting examples of peripheral vascular diseases or disorders include gangrene, diabetic vasculopathy, ischemic bowel disease, thrombosis, diabetic retinopathy and diabetic nephropathy.

Non-limiting examples of autoimmune diseases or disorders include all of the diseases caused by an immune response such as an autoantibody or cell-mediated immunity to an autoantigen and the like. Representative examples are chronic rheumatoid arthritis, juvenile rheumatoid arthritis, systemic lupus erythematosus, scleroderma, mixed connective tissue disease, polyarteritis nodosa, polymyositis/dermatomyositis, Sjogren's syndrome, Bechet's disease, multiple sclerosis, autoimmune diabetes, Hashimoto's disease, psoriasis, primary myxedema, pernicious anemia, myasthenia gravis, chronic active hepatitis, autoimmune hemolytic anemia, idiopathic thrombocytopenic purpura, uveitis, vasculitides and heparin induced thrombocytopenia.

Non-limiting examples of inflammatory glandular diseases or disorders include pancreatic diseases or disorders, Type I diabetes, thyroid diseases or disorders, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome.

Non-limiting examples of inflammatory gastrointestinal diseases or disorders include colitis, ileitis, Crohn's disease, chronic inflammatory intestinal disease, inflammatory bowel syndrome, inflammatory bowel disease, celiac disease, ulcerative colitis, an ulcer, a skin ulcer, a bed sore, a gastric ulcer, a peptic ulcer, a buccal ulcer, a nasopharyngeal ulcer, an esophageal ulcer, a duodenal ulcer and a gastrointestinal ulcer.

Non-limiting examples of inflammatory cutaneous diseases or disorders include acne, an autoimmune bullous skin disease, pemphigus vulgaris, bullous pemphigoid, pemphigus foliaceus, contact dermatitis and drug eruption.

Non-limiting examples of inflammatory hepatic diseases or disorders include autoimmune hepatitis, hepatic cirrhosis, and biliary cirrhosis.

Non-limiting examples of inflammatory neurological diseases or disorders include multiple sclerosis, Alzheimer's disease, Parkinson's disease, myasthenia gravis, motor neuropathy, Guillain-Barre syndrome, autoimmune neuropathy, Lambert-Eaton myasthenic syndrome, paraneoplastic neurological disease or disorder, paraneoplastic cerebellar atrophy, non-paraneoplastic stiff man syndrome, progressive cerebellar atrophy, Rasmussen's encephalitis, amyotrophic lateral sclerosis, Sydeham chorea, Gilles de la Tourette syndrome, autoimmune polyendocrinopathy, dysimmune neuropathy, acquired neuromyotonia, arthrogryposis multiplex, Huntington's disease, AIDS associated dementia, amyotrophic lateral sclerosis (AML), multiple sclerosis, stroke, an inflammatory retinal disease or disorder, an inflammatory ocular disease or disorder, optic neuritis, spongiform encephalopathy, migraine, headache, cluster headache, and stiff-man syndrome.

Non-limiting examples of inflammatory connective tissue diseases or disorders include autoimmune myositis, primary Sjogren's syndrome, smooth muscle autoimmune disease or disorder, myositis, tendinitis, a ligament inflammation, chondritis, a joint inflammation, a synovial inflammation, carpal tunnel syndrome, arthritis, rheumatoid arthritis, osteoarthritis, ankylosing spondylitis, a skeletal inflammation, an autoimmune ear disease or disorder, and an autoimmune disease or disorder of the inner ear.

Non-limiting examples of inflammatory renal diseases or disorders include autoimmune interstitial nephritis and/or renal cancer.

Non-limiting examples of inflammatory reproductive diseases or disorders include repeated fetal loss, ovarian cyst, or a menstruation associated disease or disorder.

Non-limiting examples of inflammatory systemic diseases or disorders include systemic lupus erythematosus, systemic sclerosis, septic shock, toxic shock syndrome, and cachexia.

Non-limiting examples of infectious disease or disorder include chronic infectious diseases or disorders, a subacute infectious disease or disorder, an acute infectious disease or disorder, a viral disease or disorder, a bacterial disease or disorder, a protozoan disease or disorder, a parasitic disease or disorder, a fungal disease or disorder, a mycoplasma disease or disorder, gangrene, sepsis, a prion disease or disorder, influenza, tuberculosis, malaria, acquired immunodeficiency syndrome, and severe acute respiratory syndrome.

Non-limiting examples of inflammatory transplantation-related diseases or disorders include graft rejection, chronic graft rejection, subacute graft rejection, acute graft rejection hyperacute graft rejection, and graft versus host disease or disorder. Exemplary implants include a prosthetic implant, a breast implant, a silicone implant, a dental implant, a penile implant, a cardiac implant, an artificial joint, a bone fracture repair device, a bone replacement implant, a drug delivery implant, a catheter, a pacemaker, an artificial heart, an artificial heart valve, a drug release implant, an electrode, and a respirator tube.

Non-limiting examples of inflammatory tumors include a malignant tumor, a benign tumor, a solid tumor, a metastatic tumor and a non-solid tumor.

Non-limiting examples of inflammatory pulmonary diseases or disorders include asthma, allergic asthma, emphysema, chronic obstructive pulmonary disease or disorder, sarcoidosis and bronchitis.

An example of a proliferative disease or disorder is cancer.

As used herein the term “about” refers to ±10%.

The terms “comprises”, “comprising”, “includes”, “including”, “having” and their conjugates mean “including but not limited to”.

The term “consisting of” means “including and limited to”.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Experimental Methods

Plasma Concentration Measurements:

Determination of VB-201 concentrations in plasma was performed using an HPLC-MS/MS (high performance liquid chromatography with tandem mass spectrometric detection) assay. Calibration was for a range of from 5 to 500 ng/ml for low doses and from 50 to 5000 ng/ml for high doses.

Pharmacokinetics Analysis:

Pharmacokinetic parameters were estimated by using a non-compartmental method, except for the AUC (area under curve), which was determined using a (log-) trapezoidal rule as the area above the previous trough level.

All calculations were made using PKWizard (developed by Russell Barbare and Dr. Shayne Gad), which was validated against WinNonlin (Pharsight Corp.), which is an FDA-recognized validated program. Pharmacokinetic parameters were calculated by the program according to standard methods used in the art.

Calculation of the pharmacokinetic characteristics was based on the actual blood sampling time (in hours), with predose times set to zero. Concentrations below the LLOQ (lower limit of quantification; 4.974 ng/ml) were assumed to be zero.

High Sensitivity C-Reactive Protein (hsCRP) Assay:

C-reactive protein (CRP) levels in plasma samples were measured using a CRP Latex assay kit (Olympus) and an AU400® immuno-analyzer (Olympus).

Cytokine Assay:

Plasma levels of interleukin-23 (IL-23) and interferon-γ (IFN-γ) were determined using the FlowCytomix human simplex kits (Bender Medsystems) for the aforementioned cytokines according to the manufacturer instructions. Acquisition of samples was performed using FacsCalibur (Becton Dickinson). Results were analyzed with the FlowCytomix Pro 2.2 Software.

Migration Assay:

Human monocytes (CD14⁺ cells) were isolated from whole blood of healthy donors. Following PBMC (peripheral blood mononuclear cell) separation from whole blood using Ficoll-Paque and leucosep tubes, CD14⁺ cells were separated using specific CD14 micro beads (Miltenyi Biotech). Purity of the CD14⁺ population was validated using specific monoclonal CD14 antibody by FACS analysis. For chemoattractant, RANTES (100 ng/ml), MIP-1α (macrophage inflammatory protein-1a; 50 ng/ml), MCP-1 (monocyte chemotactic protein-1; 50 ng/ml) or MCP-3 (50 ng/ml) were dissolved in 2% fetal bovine serum/RPMI-1640 and placed at the lower chamber of QCM™ 24-well migration assay plate (Corning-Costar). CD14+ cells were pre-incubated for 30 minutes with solvent (1% ethanol/PBS) or VB-201 at 8 μM (or different concentrations ranging between 1.7-17 μM, not shown). Migration assay was conducted by seeding 300,000 treated cells in the upper chamber, followed by incubated for 3-4 hours, after which the number of migrated cells was determined by FACS (fluorescence activated cell sorting).

Safety and Tolerability Evaluation:

Safety and tolerability of VB-201 was judged by evaluating the incidence of abnormal findings in the following measurements:

vital signs: systolic and diastolic blood pressure, heart rate, respiratory rate, body temperature;

physical examination;

12-lead electrocardiogram;

laboratory tests of blood samples for: complete blood count, prothrombin time/partial thromboplastin time, LDL- and HDL-cholesterol, total cholesterol, triglycerides, glucose, blood urea nitrogen, sodium, potassium, creatinine, calcium, phosphorus, creatinine phosphokinase, lactate dehydrogenase, aspartate transaminase, alanine transaminase, γ-glutamyl transferase, alkaline phosphatase, protein, albumin, total bilirubin, total IgM and IgG antibodies, interleukin-12/23 p40, tumor necrosis factor-α, and interleukin-6, as well as routine urinalysis; and

all adverse events, whether or not considered serious, and whether or not considered related to the investigational agent, according to the criteria for Good Clinical Practice.

Treatment-emergent adverse events were defined as any adverse event that started on or after administration of the first dose of VB-201, and no more than 30 days after administration of the last dose, or are ongoing at the time of the first dose and worsened in severity or frequency during treatment.

### Example 1

### Safety, Tolerability and Pharmacokinetics of Single Dose of VB-201

Study Design:

A randomized, double-blind, placebo-controlled study was performed to in order to investigate the safety, tolerability and pharmacokinetics of VB-201 at a single dose taken orally.

All subjects were healthy male volunteers. Five cohorts of six subjects each received a single dose of 1, 3, 10, 30 or 50 mg VB-201. 10 subjects received a placebo. The VB-201 was formulated in gelatin capsules enterocoated with Eudragit®.

Blood samples were taken prior to dosing and 0.25, 0.5, 1, 1.5, 2, 2.5, 3, 3.5, 4, 4.5, 5, 6, 7, 8, 10, 12, 16, 48, 72 and 168 hours post-dose.

Clinical Pharmacokinetics:

Mean plasma concentrations of VB-101 over time are shown in FIG. 1.

Various pharmacokinetic parameters were calculated from the data, and are presented in Table 1 below. Evaluation of relative bioavailability was performed for the primary target parameters AUC (area under the concentration−time curve) and Cₘₐₓ−(maximum plasma concentration).

The log-transformed values of the primary target parameters were subject to an analysis of variance (ANOVA) model with the effects: sequence, subjects within sequence, period and treatment. Based on the ANOVA, 90% confidence intervals for the treatment were calculated.

TABLE 1
Pharmacokinetic parameters of VB-201 in plasma following a single-dose administration
of 1, 3, 10, 30, and 50 mg VB-201 (results are presented as mean ± standard deviation, with range
in parentheses)

| | 1 mg | 3 mg | 10 mg | 30 mg | 50 mg |
|---|---|---|---|---|---|
| AUC(0-168) | 501 ± 241 | 2575 ± 1027 | 6138 ± 2259 | 23243 ± 7277 | 58816 ± 31297 |
| hours*ng/ml | (230-888) | (1512-4206) | (3105-8751) | (12084-31975) | (30284-100174) |
| AUC(0-last non- | 388 ± 168 | 2234 ± 1259 | 5634 ± 2705 | 23243 ± 7277 | 58816 ± 31297 |
| BLQ) | (158-626) | (1134-4206) | (2408-8751) | (12084-31975) | (30284-100174) |
| hours*ng/ml | | | | | |
| AUC(0-infinity) | 840 ± 309 | 2886 ± 1262 | 6395 ± 2551 | 24668 ± 7941 | 65628 ± 37141 |
| | (336-1141) | (1591-4699) | (3083-9232) | (12453-34104) | (31956-119994) |
| Cₘₐₓ | 12.52 ± 2.42 | 53.21 ± 19.43 | 131.7 ± 20.47 | 495.0 ± 154.2 | 1172 ± 459.4 |
| ng/ml | (9.98-15.83) | (35.88-90.10) | (88.91-163.5) | | (735.1-1777) |
| C_{avg} (0-168) | 2.98 ± 1.43 | 15.33 ± 6.11 | 36.53 ± 13.44 | 138.4 ± 43.32 | 350.1 ± 186.3 |
| ng/ml | (1.37-5.29) | (9.00-25.03) | (18.48-52.09) | (71.93-190.3) | (180.3-596.3) |
| T_{1/2} | 51 ± 19 | 48 ± 10 | 37 ± 5 | 39 ± 4 | 47 ± 9 |
| hours | (21-65) | (38-66) | (31-46) | (32-44) | (38-61) |
| Tₘₐₓ | 8.0 ± 2.1 | 8.2 ± 3.9 | 7.1 ± 1.9 | 6.0 ± 0.9 | 6.5 ± 0.8 |
| hours | (5-10) | (6-16) | (4.5-10) | (5-7) | (6-8) |
| AUMC | 70178 ± 38633 | 214719 ± 133999 | 333614 ± 227308 | 1463889 ± 520387 | 4719948 ± 3532459 |
| hours²*ng/ml | (11639-105421) | (91371-412681) | (66795-623656) | (656277-2030839) | (1727351-10830296) |
| Kₑ | 0.0165 ± 0.0099 | 0.0150 ± 0.0027 | 0.0191 ± 0.0026 | 0.0180 ± 0.0021 | 0.0150 ± 0.0027 |
| liters/hour | (0.0106-0.0337) | (0.0104-0.0181) | (0.0151-0.0222) | (0.0158-0.0216) | |
| MTT | 76 ± 27 | 70 ± 16 | 46 ± 20 | 59 ± 4 | 67 ± 14 |
| hours | (35-96) | (56-99) | (22-68) | (53-65) | (52-90) |
| Cl/F | 1.44 ± 0.087 | 1.21 ± 0.49 | 1.83 ± 0.84 | 1.37 ± 0.58 | 0.97 ± 0.46 |
| liters/hour | (0.88-2.97) | (0.64-1.89) | (1.08-3.24) | (0.88-2.41) | (0.42-1.56) |
| Vz/F | 88 ± 10 | 79 ± 21 | 93 ± 32 | 74 ± 22 | 63 ± 25 |
| liters | (76-102) | (47-109) | (60-151) | (51-111) | (30-92) |
| Estimated amount | 0.26 ± 0.05 | 1.12 ± 0.41 | 2.77 ± 0.62 | 10.40 ± 3.24 | 24.61 ± 9.65 |
| circulating | (0.21-0.33) | (0.75-1.89) | (1.87-3.43) | (5.53-13.86) | (15.44-37.32) |
| mg | | | | | |
| Estimated minimum | 26 ± 5 | 37 ± 14 | 28 ± 6 | 35 ± 11 | 49 ± 19 |
| bioavailability % | (21-33) | (25-63) | (19-34) | (18-46) | (31-75) |

AUC(0-168) = area under concentration-time curve from 0 hours to 168 hours
AUC(0-last non-BLQ) = area under concentration time curve from 0 hours to last measurement which was not below the limit of quantification
AUC(0-infinity) = area under concentration-time curve from 0 hours to infinity
Cₘₐₓ = maximum plasma concentration
C_{avg}(0-168) = average concentration from 0 hours to 168 hours
T_{1/2} = elimination half-life
Tₘₐₓ = time until maximum plasma concentration is reached
AUMC = area under the moment curve of the concentration-time curve
MTT = mean transit time
Kₑ = elimination rate constant
Cl/F = clearance
Vz/F = apparent volume of distribution during terminal phase after oral administration

As shown in FIG. 1 and Table 1, the pharmacokinetics of VB-201 was dose-linear and nearly dose-proportional with regard to AUC (area under concentration-time curve) and Cₘₐₓ (maximum plasma concentration).

As further shown therein, Tₘₐₓ values were relatively independent of dose levels (ranging from 6 to 8 hours). VB-201 plasma levels rose generally linearly until Tₘₐₓ was reached. The mean half life (T_{1/2}) was 37 to 51 hours.

As further shown in Table 1, the clearance parameters mean transit time (MTT), apparent clearance (Cl/F), and apparent volume of distribution (Vz/F) were relatively independent of dose levels.

These results are consistent with the approximately linear uptake and single compartment kinetics during the elimination phase, indicating that single compartment kinetics are a good approximation for the pharmacokinetics of VB-201 over the range of doses used in this study.

The lowest and highest estimated bioavailability values observed in individuals were 18% and 75%, respectively. Bioavailability values were similar over the range of doses studied, although the highest values (49% mean) were observed at a dose of 50 mg. 2 subjects receiving a 50 mg dose had particularly high values for estimated bioavailability (75% and 71% compared to an average of 38% for the other 4 subjects in the same cohort). Without these 2 subjects, the average for the 50 mg cohort would have been similar to that for the other cohorts.

Given that these bioavailability calculations only include the amount in the extracellular fluid compartment, which is estimated at approximately 30% of body mass, the relatively high numbers could indicate either high absorption, a strong tendency towards compartmentalization in the extracellular fluid compartment, or some combination of the two.

Safety and Tolerability:

Safety and tolerability was determined as described hereinabove.

No serious adverse events occurred in any treatment group.

83 treatment-emergent adverse events were reported in 27 out of 40 subjects. 32 treatment-emergent adverse events in 12 subjects were considered drug-related. All treatment-emergent adverse events were of mild or moderate intensity and no event was of severe intensity. The incidence of adverse events did not appear to be dose-related.

Cut-offs to list laboratory abnormalities have been set arbitrarily to report only those abnormalities≧1.5× upper limit of normal (ULN) and ≦0.8× lower limit of normal (LLN) to exclude “background noise” and to only concentrate on laboratory parameter changes of a certain magnitude. Most abnormalities were transient, clinically irrelevant, and gave no indication for drug-induced laboratory parameter changes. The laboratory abnormalities did not indicate clinically relevance.

Adverse events and laboratory abnormalities were even less frequent in the 2 subjects with very high VB-201 exposure compared to the other subjects.

No substantial changes were detected in immune cell response, serum antibodies, and cytokines levels.

Changes in vital signs (heart rate, systolic and diastolic blood pressure, body temperature, and respiratory rate) were not clinically significant.

None of the subjects which received VB-201 had clinically significant electrocardiogram findings. One case of sinus tachycardia associated with the occurrence of fever was found in a subject from the placebo group.

These results indicated a comparable safety profile of VB-201 to a placebo.

### Example 2

### Safety, Tolerability and Pharmacokinetics of Repeated Doses of VB-201

Study Design:

A randomized, double-blind, placebo-controlled study was performed to in order to investigate the safety, tolerability and pharmacokinetics of VB-201 at a repeated, daily dose taken orally. The subjects were all healthy volunteers. Cohorts of six subjects each received a daily dose 5, 10, 20 and 30 mg VB-201 in the morning, following at least 10 hours of fasting, for 14 days. 10 subjects received a placebo. The VB-201 was formulated in gelatin capsules enterocoated with Opadry AMB and Acryleze White.

The 20 mg dose was originally in the form of 20 mg capsules. Due to significantly reduced absorption and delayed uptake when using 20 mg capsules, the data were invalidated and the 20 mg cohort was repeated using administration of four 5 mg capsules to new subjects.

Pharmacokinetic blood samples for VB-201 determination were collected on Day 0 and Day 14, prior to dosing and 2, 4, 6, 8, 10, 12, 16 and 24 hours after dosing. Blood samples were also taken 48, 72, 96, 120, 144, 168 and 192 hours after VB-201 administration on day 14 (i.e., on days 16-22). Pharmacokinetic samples were also collected on Day 12 and Day 13, before dosing.

Overall compliance was very good with 99.4% to 100% of expected study drug intake.

Clinical Pharmacokinetics:

Concentration-time curves were constructed for a time period of 24 hours on each of days 1 and 14. Mean plasma concentrations of VB-101 during days 1 and 14 are shown in FIG. 2.

Various pharmacokinetic parameters were calculated from the data, and are presented in Table 2 below.

TABLE 2
Pharmacokinetic parameters of VB-201 in plasma on days 1 and 14 following daily administration
of 5, 10, 20 and 30 mg VB-201 (results are presented as: mean ± standard deviation)

| | 5 mg | | 10 mg | | 20 mg | | 30 mg | |
|---|---|---|---|---|---|---|---|---|
| | Day 1 | Day 14 | Day 1 | Day 14 | Day 1 | Day 14 | Day 1 | Day 14 |
| AUC(0-24) | 1731 ± | 6238 ± | 3494 ± | 12617 ± | 4186 ± | 20553 ± | 6409 ± | 30555 ± |
| hours*ng/ml | 815 | 2363 | 1682 | 3788 | 1435 | 9341 | 3247 | 10739 |
| AUC | 6005 ± | 15913 ± | 9912 ± | 36183 ± | 12721 ± | 47828 ± | 67386 ± | 64120 ± |
| (0-infinity) | 2717 | 7107 | 2782 | 28897 | 7032 | 27941 | 74614 | 21886 |
| hours*ng/ml | | | | | | | | |
| Cₘₐₓ | 117.34 ± | 340.31 ± | 227.92 ± | 665.80 ± | 294.23 ± | 1275.65 ± | 416.58 ± | 1821.40 ± |
| ng/ml | 37.62 | 122.14 | 80.36 | 189.59 | 110.73 | 574.51 | 202.82 | 727.99 |
| Tₘₐₓ | 10 ± | 9.67 ± | 11.33 ± | 11 ± | 12.33 ± | 7.33 ± | 19 ± | 10 ± |
| hours | 7.04 | 7.53 | 7.23 | 7.56 | 9.07 | 2.73 | 7.97 | 7.69 |
| AUMC | 23670 ± | 71173 ± | 45924 ± | 148375 ± | 58323 ± | 229287 ± | 92749 ± | 358861 ± |
| (0-24) | 10104 | 27589 | 20238 | 45976 | 18564 | 102760 | 47518 | 121334 |
| hours²*ng/ml | | | | | | | | |
| AUMC | 341432 ± | 821875 ± | 440602 ± | 2596242 ± | 752013 ± | 2070644 ± | 11648110 ± | 2456341 ± |
| (0-infinity) | 236622 | 650679 | 259681 | 3551596 | 843032 | 1614473 | 15845277 | 1351432 |
| hours²*ng/ml | | | | | | | | |
| MTT | 14.51 ± | 11.41 ± | 14.44 ± | 11.72 ± | 14.04 ± | 11.16 ± | 14.73 ± | 11.78 ± |
| hours | 3.28 | 0.53 | 4.01 | 0.48 | 1.83 | 0.28 | 1.39 | 0.76 |
| Cl/F(0-24) | 3.92 ± | 0.90 ± | 4.92 ± | 0.87 ± | 5.21 ± | 1.46 ± | 8.55 ± | 1.09 ± |
| liters/hour | 3.03 | 0.33 | 5.77 | 0.32 | 1.59 | 1.45 | 10.49 | 0.39 |
| Vd/F | 120.84 ± | 40.98 ± | 105.40 ± | 40.64 ± | 205.91 ± | 47.71 ± | 316.00 ± | 39.39 ± |
| liters | 47.16 | 38.25 | 61.73 | 18.45 | 154.58 | 40.51 | 302.96 | 22.14 |
| K_{c} | 0.0250 ± | 0.0278 ± | 0.0285 ± | 0.0267 ± | 0.0307 ± | 0.0338 ± | 0.0220 ± | 0.0333 ± |
| hours⁻¹ | 0.0112 | 0.0123 | 0.0097 | 0.0122 | 0.0163 | 0.0196 | 0.0234 | 0.0128 |
| T_{1/2} | 32.18 ± | 30.95 ± | 27.09 ± | 34.27 ± | 30.91 ± | 25.09 ± | 72.30 ± | 23.64 ± |
| hours | 13.25 | 17.71 | 10.98 | 24.82 | 23.61 | 10.53 | 76.83 | 9.27 |
| Accum. | NA | 4.75 ± | NA | 5.06 ± | NA | 4.90 ± | NA | 7.07 ± |
| | | 4.16 | | 4.23 | | 1.94 | | 6.64 |
| T_{1/2} | NA | 44.17 ± | NA | 35.77 ± | NA | 30.89 ± | NA | 32.78 ± |
| (rec. ph.) | | 6.82 | | 8.87 | | 6.66 | | 3.37 |
| hours | | | | | | | | |

AUC(0-24) = area under concentration-time curve from 0 hours to 24 hours after administration
AUC(0-infinity) = area under concentration-time curve from 0 hours to infinity
Cₘₐₓ = maximum plasma concentration
Tₘₐₓ = time until maximum plasma concentration is reached
T_{1/2} = elimination half-life
T_{1/2} (rec. ph.) = elimination half-life during recovery phase
AUMC(0-24) = area under the moment curve of the concentration-time curve from 0 hours to 24 hours after administration
AUMC(0-infinity) = area under the moment curve of the concentration-time curve from 0 hours to infinity
MTT = mean transit time
Kₑ = elimination rate constant
Cl/F(0-24) = clearance from 0 to 24 hours after administration
Vd/F = volume of distribution
Accum. = accumulation
NA = not applicable

As shown in FIG. 2 and Table 2, AUC (area under concentration-time curve) and Cₘₐₓ (maximum plasma concentration) values were generally linearly correlated with dosage over the dose range of 5 to 10 mg, but were slightly lower for 20 mg and 30 mg dosages than would be expected from a purely linear correlation.

This variation is possibly a result of gastric emptying following ingestion of respectively four 5 mg capsules (for a 20 mg dosage) and six 5 mg capsules (for a 30 mg dosage).

As further shown in Table 2, the average accumulation rate was approximately 5, but was slightly higher for a 30 mg dosage.

Terminal elimination half-lives could not be reliably calculated over the 0-24 hour range because of the long T_{1/2} for the compound. More reliable measurements were calculated from the recovery phase (days 14-22).

As shown in Table 2, there was a slight downward trend in T_{1/2} with increased dosage. Estimations based on these elimination data show that continued dosing on a once per day schedule would generate a multi-day accumulation of approximately 4 times that of a single dose, which is consistent with the observed range of accumulation.

As further shown in Table 2, the clearance parameters Cl/F (clearance) and MIT (mean transit time) were relatively independent of dose. MTT values were similar for all dosage levels tested. Clearance values at dosages of 5 mg and 10 mg were similar (especially when 2 outliers were discounted). Clearance values were somewhat higher at dosages of 20 and 30 mg. The differences between Day 1 values and Day 14 values may be artifacts of the long half-lives, which affect both calculations.

The lack of dose effect on clearance and MIT indicates that VB-201 does not show significant induction or inhibition of metabolism over the range of doses studied and that the doses studied were not near metabolic saturation.

In addition, urine samples from the subjects receiving 30 mg VB-201 were assayed for VB-201 by high performance liquid chromatography-tandem mass spectrometry. No traces of VB-201 could be detected in any of the urine samples. The limit of detection was 1 ng/ml.

Safety and Tolerability:

Safety and tolerability was determined as described hereinabove.

No serious or severe adverse events occurred.

An adverse event occurred for one subject in each of the placebo group, 10 mg group and 20 mg group. All adverse events were considered to be unrelated to VB-201.

Laboratory assessments (hematology, blood chemistry, fasting lipid parameters and urinalysis), vital signs (BP, HR, temperature and RR), electrocardiograms and physical examinations did not result in any report of an adverse effect and did not reveal any safety concerns.

These results indicate that VB-201 administered orally for 14 days at daily doses ranging from 5 mg to 30 mg is as safe and tolerable as placebo.

### Example 3

### Effects of Food and Capsule Coating on Safety, Tolerability and Pharmacokinetics of Single Dose of VB-201

Study Design:

The effects of food on the pharmacokinetics of enteric coated and non-coated VB-201 capsules were investigated in a double-blind, placebo-controlled study in healthy subjects. Cohorts of six subjects each received a single dose of 10 mg, 2×10 mg or 20 mg VB-201 in the morning. For each dosage level, one cohort received enteric coated capsules and one cohort received non-coated capsules. 12 subjects received a placebo.

Subjects were randomly assigned to receive the drug when fed (i.e., following completion of a standardized high fat meal) or when fasting (i.e., fasting for at least 10 hours). Blood samples were drawn 2, 3, 4, 5, 6, 8, 10, 12, 14, 16, 18, 20, 24, 28, 48, 72, 96, 120 and 144 hours post-dose. Subjects then underwent an additional 8 day washout period, after which subjects underwent a second administration. Subjects who received the first administration when fasting, received the second administration when fed, and subjects who received the first administration when fed, received the second administration when fasting. Following the second administration, blood samples were taken at the same times post-dose as previously.

All subjects completed the study, except for one member of the placebo group.

Clinical Pharmacokinetics:

Concentration-time curves were constructed for time periods of up to 144 hours. Pharmacokinetic parameters were calculated based on data for the first 24 hours and for the whole 144 hours. Parameters calculated from data covering 144 hours provide a more accurate estimate of metabolic parameters. Nevertheless, parameters were also calculated from data covering the first 24 hours in order to compare parameters with previous studies.

Statistical comparisons of doses, coated vs. non-coated capsules, feeding vs. fasting and coating-feeding interactions were performed using a mixed model ANOVA in an SAS 8.02 software package (SAS Institute, Cary, N.C.) using PROC MIXED

Mean plasma concentrations of VB-101 during the first 24 hours are shown in FIG. 3.

Various pharmacokinetic parameters calculated from the data are presented in Table 3 below.

TABLE 3
Effect of fasting and enteric coating on mean pharmacokinetic parameters of VB-
201 in plasma following single administration of 10, 2 × 10 and 20 mg VB-201

| | 1 × 10 mg | | | | 1 × 20 mg [2 × 10 mg] |
|---|---|---|---|---|---|
| | coated fasting | coated fed | non-coated fasting | non-coated fed | coated fasting |
| AUC(0-24) hours*ng/ml | 2602 | 2202 | 5759 | 4855 | 5362 [6427] |
| AUC(0-144) hours*ng/ml | 7397 | 8583 | 14544 | 13717 | 14579 [20269] |
| AUC (0-infinity, from 24) hours*ng/ml | 4801 | 5898 | 9375 | 7019 | 9794 [10388] |
| AUC (0-infinity, from 144) hours*ng/ml | 8017 | 9751 | 15995 | 16062 | 15637 [24950] |
| Cₘₐₓ ng/ml | 171.4 | 186.4 | 364.5 | 343.3 | 366.3 [424.6] |
| Tₘₐₓ hours | 9.33 | 13.67 | 7.67 | 10.17 | 8.17 [9.67] |
| AUMC (0-24) hours²*ng/ml | 33556 | 32798 | 67186 | 60611 | 68472 [83132] |
| AUMC (0-infinity) hours²*ng/ml | 467760 | 699311 | 924243 | 1254665 | 849853 [2246560] |
| MTT (0-24) hours | 12.8 | 15.1 | 11.7 | 12.5 | 12.8 [12.9] |
| MTT (from 144) hours | 60.7 | 70.7 | 54.2 | 70.3 | 53.8 [69.2] |
| Cl/F (0-24) liters/hour | 3.293 | 1.883 | 1.184 | 1.552 | 3.188 [3.318] |
| Cl/F (from 144) liters/hour | 1.580 | 1.091 | 0.695 | 0.702 | 2.387 [1.815] |
| Vd/F (0-24) liters | 75.5 | 57.8 | 21.4 | 23.7 | 101.3 [65.0] |
| Vd/F (from 144) liters | 92.1 | 70.0 | 36.5 | 43.1 | 105.9 [89.5] |
| Kₑ (from 24) hours⁻¹ | 0.0438 | 0.0352 | 0.0575 | 0.0670 | 0.0385 [0.0493] |
| Kₑ (from 144) hours⁻¹ | 0.0201 | 0.0166 | 0.0190 | 0.0180 | 0.0203 [0.0174] |
| T_{1/2} (from 24) hours | 17.8 | 23.4 | 13.6 | 11.2 | 19.9 [14.9] |
| T_{1/2} (from 144) hours | 37.6 | 45.0 | 39.7 | 47.7 | 35.6 [48.0] |

| | 1 × 20 mg [2 × 10 mg] | | |
|---|---|---|---|
| | coated fed | non-coated fasting | non-coated fed |
| AUC(0-24) hours*ng/ml | 4655 [5008] | 10013 [10036] | 8334 [8708] |
| AUC(0-144) hours*ng/ml | 14968 [21417] | 26650 [24028] | 24282 [24104] |
| AUC (0-infinity, from 24) hours*ng/ml | 20054 [15799] | 16958 [14643] | 23552 [15305] |
| AUC (0-infinity, from 144) hours*ng/ml | 16687 [24463] | 29478 [25734] | 26424 [25822] |
| Cₘₐₓ ng/ml | 306.6 [422.3] | 660.4 [724.4] | 546.4 [652.1] |
| Tₘₐₓ hours | 11.67 [14.33] | 8.33 [8.67] | 8.17 [8.50] |
| AUMC (0-24) hours²*ng/ml | 66330 [74749] | 122207 [117485] | 111005 [109878] |
| AUMC (0-infinity) hours²*ng/ml | 1070977 [1794144] | 1763607 [1319516] | 1555946 [1409500] |
| MTT (0-24) hours | 14.9 [15.1] | 12.0 [11.7] | 13.0 [12.7] |
| MTT (from 144) hours | 62.9 [64.6] | 61.0 [51.5] | 57.2 [54.3] |
| Cl/F (0-24) liters/hour | 1.400 [2.067] | 1.301 [1.444] | 1.197 [1.394] |
| Cl/F (from 144) liters/hour | 1.583 [1.239] | 0.702 [0.810] | 0.852 [0.802] |
| Vd/F (0-24) liters | 82.9 [54.2] | 27.2 [24.8] | 35.2 [28.8] |
| Vd/F (from 144) liters | 88.0 [60.8] | 44.8 [42.7] | 44.4 [41.8] |
| Kₑ (from 24) hours⁻¹ | 0.0230 [0.0427] | 0.0486 [0.0606] | 0.0332 [0.0528] |
| Kₑ (from 144) hours⁻¹ | 0.0179 [0.0196] | 0.0166 [0.0195] | 0.0193 [0.0195] |
| T_{1/2} (from 24) hours | 53.2 [22.8] | 14.6 [11.7] | 25.2 [15.4] |
| T_{1/2} (from 144) hours | 39.6 [37.7] | 43.5 [36.3] | 36.4 [36.1] |

AUC(0-24) = area under concentration-time curve from 0 hours to 24 hours after administration
AUC(0-144) = area under concentration-time curve from 0 hours to 144 hours after administration
AUC(0-infinity, from 24) = area under concentration-time curve from 0 hours to infinity, as calculated based on data from 0 to 24 hours
AUC(0-infinity, from 144) = area under concentration-time curve from 0 hours to infinity, as calculated based on data from 0 to 144 hours
Cₘₐₓ = maximum plasma concentration
Tₘₐₓ = time until maximum plasma concentration is reached
T_{1/2} (from 24) = elimination half-life calculated based on data from 0 to 24 hours
T_{1/2} (from 144) = elimination half-life calculated based on data from 0 to 144 hours
AUMC(0-24) = area under the moment curve of the concentration-time curve from 0 hours to 24 hours after administration
AUMC(0-infinity) = area under the moment curve of the concentration-time curve from 0 hours to infinity (calculated based on data from 0 to 144 hours)
MTT (0-24) = mean transit time calculated over 0 to 24 hours
MTT (from 144) = mean transit time calculated based on data from 0 to 144 hours
Kₑ (from 24) = elimination rate constant calculated based on data from 0 to 24 hours
Kₑ (from 144) = elimination rate constant calculated based on data from 0 to 144 hours
Cl/F (0-24) = clearance calculated over 0 to 24 hours after administration
Cl/F (from 144) = clearance calculated based on data from 0 to 144 hours
Vd/F (0-24) = volume of distribution over 0 to 24 hours after administration
Vd/F (from 144) = volume of distribution calculated based on data from 0 to 144 hours
Vd/F (from 144) = volume of distribution calculated based on data from 0 to 144 hours

As shown in FIG. 3 and Table 3, AUC and Cₘₐₓ values were proportional to the dose, indicating a linear dose response.

As further shown therein, coated capsules significantly lowered AUC and Cₘₐₓ values.

With respect to AUC and Cₘₐₓ values, there were no statistically significant differences observed between fed and fasting subjects. In addition, there were no statistically significant coating-feeding interactions.

As further shown in Table 3, enteric coatings increased Tₘₐₓ. However, fasting subjects exhibited a considerably smaller coating-induced increase in Tₘₐₓ than did fed subjects.

As further shown therein, terminal elimination half-lives (T_{1/2}) and mean transit times (MTT) were not affected by dose level, coating or feeding.

However, both T_{1/2} and MIT values were higher when calculated over the 0 to 144 hour time period than when calculated over the 0 to 24 hour time period. These results indicate that metabolism of VB-201 is not consistent with first-order kinetics.

None of the tested pharmacokinetic parameters were significantly affected by whether subjects received one 20 mg capsule or two 10 mg capsules.

Safety and Tolerability:

Safety and tolerability was determined as described hereinabove.

No serious or treatment-emergent adverse events occurred. The overall incidence of adverse effects was low (5 adverse effects in 4 subjects), all adverse effects were mild and considered unrelated to the study medication.

No adverse effects related to laboratory values, vital signs, electrocardiograms or physical examination were reported.

These results indicate that enteric coated and non-coated capsules with 10 to 20 mg VB-201 are as safe and tolerable as placebo, whether taken while fasting or following feeding.

### Example 4

### Safety, Tolerability, Pharmacokinetics and Effect on C-Reactive Protein Levels of Repeated Increased Doses of VB-201

Study Design:

A randomized, double-blind, placebo-controlled study was performed to in order to investigate the safety, tolerability and pharmacokinetics of VB-201 at a repeated, daily dose taken orally. The subjects were all healthy volunteers. Cohorts of six subjects each received a daily dose of 40 or 80 mg VB-201 in the morning, following at least 10 hours of fasting, for 14 days. 6 subjects received a placebo. The VB-201 was formulated in non-enterocoated capsules.

Further studies were performed on an additional cohort, which included six subjects receiving a daily dose of 80 mg VB-201, as described hereinabove, for 28 days. An additional two subjects received a placebo.

Blood was collected for pharmacokinetic evaluation on day 1 (40 mg cohort) and on day 14 (40 and 80 mg cohorts).

Plasma samples obtained on day 0 (pre-dosing) and on day 14 from the subjects in each cohort (40 mg for 14 days, 80 mg for 14 days, and 80 mg for 28 days), and on day 28 from the subjects of the cohort receiving 80 mg VB-201 for 28 days, were assayed for C-reactive protein (CRP) by the high sensitivity CRP assay described hereinabove.

Clinical Pharmacokinetics:

Mean plasma concentrations of VB-201 during 24 hour time periods following administration were determined for days 1 and 14 of the 40 mg cohort, and for day 14 of the 80 mg cohort, and pharmacokinetic parameters were determined for the data representing each of these time periods. The mean plasma concentrations are shown in FIG. 4.

Various pharmacokinetic parameters calculated from the data are presented in Table 4 below.

TABLE 4
Pharmacokinetic parameters of VB-201 in plasma on days 1
and 14 following daily administration of 40 and 80 mg VB-201
(results are presented as: mean ± standard deviation)

| | 40 mg | | 80 mg |
|---|---|---|---|
| | Day 1 | Day 14 | Day 14 |
| AUC(0-24) | 1595 ± 5983 | 47393 ± 16792 | 89779 ± 21751 |
| hours*ng/ml | | | |
| AUC(0-infinity) | 31952 ± 9680 | 117013 ± 55816 | 206918 ± 62474 |
| hours*ng/ml | | | |
| Cₘₐₓ | 923.7 ± 287.4 | 2544.6 ± 800.5 | 4820.2 ± 930.3 |
| ng/ml | | | |
| Tₘₐₓ | 8.7 ± 3.8 | 4.2 ± 2.0 | 6.0 ± 1.1 |
| hours | | | |
| MTT(0-24) | 12.0 ± 0.5 | 11.1 ± 0.4 | 11.0 ± 0.4 |
| hours | | | |
| MTT(0-infinity) | 41.3 ± 17.6 | 45.2 ± 18.8 | 41.5 ± 7.8 |
| hours | | | |
| Cl/F(0-24) | 2.837 ± 1.064 | 0.970 ± 0.436 | 0.941 ± 0.247 |
| liters/hour | | | |
| Cl/F(0-infinity) | 1.346 ± 0.401 | 0.405 ± 0.172 | 0.422 ± 0.144 |
| liters/hour | | | |
| Vd/F(0-24) | 127.83 ± 99.22 | 44.72 ± 37.40 | 38.35 ± 10.57 |
| liters | | | |
| Vd/F(0-infinity) | 49.69 ± 22.44 | 16.58 ± 9.23 | 16.87 ± 4.65 |
| liters | | | |
| Kₑ | 0.0305 ± 0.0124 | 0.0270 ± 0.0111 | 0.0251 ± 0.0048 |
| hours⁻¹ | | | |
| T_{1/2} | 26.8 ± 13.0 | 30.2 ± 13.6 | 28.5 ± 5.8 |
| hours | | | |
| Accumulation | NA | 3.01 ± 0.58 | NA |

AUC(0-24) = area under concentration-time curve from 0 hours to 24 hours after administration
AUC(0-infinity) = area under concentration-time curve from 0 hours to infinity
Cₘₐₓ = maximum plasma concentration
Tₘₐₓ = time until maximum plasma concentration is reached
T_{1/2} = elimination half-life
MTT(0-24) = mean transit time calculated over 0 to 24 hours
MTT(0-infinity) = mean transit time calculated over 0 hours to infinity
Kₑ = elimination rate constant
Cl/F(0-24) = clearance calculated over 0 to 24 hours after administration
Cl/F(0-infinity) = clearance calculated over 0 hours to infinity
Vd/F(0-24) = volume of distribution calculated over 0 to 24 hours after administration
Vd/F(0-infinity) = volume of distribution calculated over 0 hours to infinity
NA = not applicable

As shown in FIG. 4 and Table 4, AUC and Cₘₐₓ values on day 14 were proportionate to the dosage level.

As further shown therein, the observed accumulation ratio between day 14 and day 1 was approximately 3 (only the 40 mg cohort was measured).

As further shown in Table 4, the elimination half-lives (T_{1/2}) and mean transit times (MIT) were independent of dosage levels.

C-Reactive Protein (CRP) Levels:

C-reactive protein (CRP) is a known marker for inflammatory processes.

As shown in FIGS. 5A, 5B and 5C, administration of 40 mg per day VB-201 for 14 days, administration of 80 mg per day VB-201 for 14 days, and administration of 80 mg per day VB-201 for 28 days, reduced plasma CRP levels on average by 49%, 43% and 45%, respectively.

These results indicate that both 40 mg VB-201 per day and 80 mg VB-201 per day effectively and quickly reduce inflammation.

Safety and Tolerability:

Safety and tolerability was determined as described hereinabove.

No serious adverse events were reported for either 14 or 28 day cohorts.

Laboratory anomalies were observed in only one subject, and these were attributed to vigorous exercise performed by the subject the previous day, rather than to VB-201, and the anomalies did not recur.

No clinically significant changes in vital signs, physical exams or electrocardiograms were observed post-dosing.

These results indicate that daily doses of up to 80 mg VB-201 for up to 28 days are both safe and well-tolerated.

### Example 5

### Effect of 80 Mg Per Day VB-201 on Cytokine Levels

Interleukin-23 (IL-23) is an important part of the inflammatory response against infection, and promotes production of numerous inflammatory molecules. IL-23 plays an important role in the development of symptoms of multiple sclerosis and inflammatory bowel disease, highlighting the importance of IL-23 in inflammatory disease pathways.

Interferon-γ (IFN-γ) is a cytokine which is expressed by Th1 cells and suppresses activity of Th2 cells. IFN-γ promotes leukocyte migration. IFN-γ expression is associated with a number of auto-inflammatory and autoimmune diseases.

Healthy subjects were treated once a day with 40 mg or 80 mg of VB-201, according to the procedures described in Example 4. Plasma was collected on day 0 (pre-dosing), on day 14 after dosing, and on day 28 after dosing (when administration lasted 28 days). Plasma levels of interleukin-23 (IL-23) and interferon-γ (IFN-γ) were determined as described hereinabove. Pre-dose levels of IL-23 and IFN-γ were very low in many subjects. Results from subjects having high pre-dose cytokine levels are presented in FIGS. 6A-6D.

As shown in FIG. 6A, administration of 40 mg per day VB-201 considerably reduced IL-23 levels in both subjects which exhibited detectable pre-dose IL-23 levels.

As shown in FIG. 6B, administration of 40 mg per day VB-201 considerably reduced IFN-γ levels in one subject, whereas the second subject had no detectable IFN-γ levels before or after administration.

As shown in FIG. 6C, administration of 80 mg per day VB-201 considerably reduced IL-23 levels in all three subjects.

As shown in FIG. 6D, administration of 80 mg per day VB-201 considerably reduced IFN-γ levels in two subjects, whereas the third subject had no detectable IFN-γ levels before or after administration.

These results indicate that VB-201 inhibits pro-inflammatory cytokines which play important roles in auto-inflammatory responses.

These results also indicate that VB-201 is effective against inflammatory diseases and disorders such as multiple sclerosis and inflammatory bowel disease.

### Example 6

### Efficacy of VB-201 with Methotrexate (MTX) in Patients with Active Rheumatoid Arthritis (RA)

VB-201 was previously shown to be effective in the in vivo animal models of adjuvant-induced arthritis and collagen-induced arthritis. VB-201 has also been shown to have immunomodulatory effects which may slow progression of rheumatoid arthritis (RA). The efficacy of VB-201 in treating RA in humans is therefore investigated.

A randomized, double blind, Phase II study is performed in subjects with active RA receiving VB-201 with concomitant methotrexate (MTX) compared to subjects receiving MTX alone.

Diagnosis of RA is based on the 1987 Revised American Rheumatism Association Criteria for the Classification of Rheumatoid Arthritis. Subjects already receiving MTX (at least 12.5 mg per week, for at least 6 months) are selected. The number of subjects enrolled with a history of previous RA treatment with biologics (e.g., TNF-α inhibitors, T-cell inhibitors, and B-cell inhibitors) is limited to 30% of all subjects.

After screening and establishment of a baseline, eligible subjects are randomly assigned to receive 20 mg per day VB-201, 80 mg per day VB-201, or placebo, for a period of 12 weeks. Doses are administered orally at breakfast time. All subjects continue to receive MTX at the same dose utilized prior to study entry.

The maximal dose of VB-201 (80 mg/day) was determined according to the tolerability results presented in Example 4.

Efficacy of each dosage level of VB-201 relative to placebo and MTX only groups is determined by measuring mean change from baseline for each of ACR components and percentage of patients achieving ACR20, ACR50 and ACR70 responses (as defined by the American College of Rheumatology) at weeks 2, 4, 8, 12 and 16.

Additional criteria include the percentage of subjects requiring rescue intervention, and the percentage of subjects achieving low disease activity (score of 3.2 or less), remission (score of <2.6), “good” response or “moderate” response, as calculated according to the Disease Activity Score 28 (DAS28) at weeks 2, 4, 8, 12 and 16.

Safety of VB-201 administration is evaluated as described hereinabove by physical examination, incidence of adverse effects, vital signs, clinical chemistry, hematology, urinalysis and electrocardiograms.

Statistical comparisons are performed using a two-sided comparison with a 5% level of significance.

### Example 7

### Efficacy of VB-201 in Patients with Plaque Psoriasis

A randomized, double-blind Phase II study is performed in subjects with moderate to severe plaque psoriasis, in order to determine the efficacy of VB-201 in treating this condition.

Patients are selected with moderate (i.e., scoring at least 3 on a 0 to 5 point Physician Global Assessment (PGA)) to severe, stable and active plaque psoriasis vulgaris affecting at least 10% of the body surface and with a Psoriasis Area and Severity Index (PASI) score of at least 12. Patients must undergo a wash-out period following any previous treatments.

After screening and establishment of a baseline, eligible subjects are randomly assigned to receive 20 mg per day VB-201, 80 mg per day VB-201 or a daily placebo, for a period of 12 weeks. Doses are administered orally at breakfast time with food.

The maximal dose of VB-201 (80 mg/day) was determined according to the tolerability results presented in Example 4.

Efficacy of each dosage level of VB-201 relative to placebo is determined according to the percentage of patients which achieve at least a 50% or 75% improvement over baseline PASI score at week 12.

Additional criteria include change in affected body surface area from baseline to week 12, change in PGA score from baseline to week 12, any change relative to baseline PASI score, and change in Patient Psoriasis Global Assessment score from baseline to week 12.

Blood samples are taken at weeks 4, 8 and 12, in order to assess VB-201 pharmacokinetics, as well as plasma cytokine levels.

Safety of VB-201 administration is evaluated as described hereinabove by physical examination, incidence of adverse effects, vital signs, clinical chemistry, hematology, urinalysis and electrocardiograms.

Statistical comparisons are performed using a two-sided comparison with a 5% level of significance.

### Example 8

### Effect of VB-201 on Carotid Artery and Ascending Aorta ¹⁸FDG Uptake in Patients with Abnormal Baseline ¹⁸FDG Scans

The effect of treatment with 20 mg per day and 80 mg per day VB-201 on carotid artery and ascending aorta inflammation is examined. Inflammation is measured by positron emission computed tomography (PET-CT) to quantify ¹⁸FDG (18F-fludeoxyglucose) uptake as a target to background ratio (TBR).

Subjects are scanned by PET-CT to determine baseline ¹⁸FDG uptake, and those with a TBR of at least 1.6 in either the carotid artery or the ascending aorta are selected for study.

After screening and establishment of a baseline, eligible subjects are randomly assigned to receive 20 mg per day VB-201, 80 mg per day VB-201 or a daily placebo, for a period of 12 weeks. Doses are administered orally at breakfast time with food.

The maximal dose of VB-201 (80 mg/day) was determined according to the tolerability results presented in Example 4.

The TBR of each subject is determined again by PET-CT at week 12 of the treatment. Efficacy is determined by comparing TBR values obtained before and after treatment.

This subjects of this study can be a subset of subjects of a second study (e.g., the study described in Example 7), and the study can be performed concomitantly with the second study.

### Example 9

### Effect of VB-201 on Monocyte Migration

Cell migration is an essential process during many phases of development and adult life. Cells can either migrate as individuals or move in the context of tissues. Movement is controlled by internal and external signals, which activate complex signal transduction cascades resulting in highly dynamic and localized remodeling of the cytoskeleton, cell-cell and cell-substrate interactions. Chemoattractants induce cell migration through the activation of a distinct family of structurally related heterotrimeric guanine nucleotide-binding protein (G protein)-coupled receptors. Chemokines or chemotactic cytokines are 8- to 10-kDa secreted proteins that regulate migration and activation of not only leukocytes, including the DC, but also of stromal cells. Chemoattraction occurs in response to a local gradient of chemokines in the extracellular space.

VB-201 was tested in vitro as described hereinabove for possible effects on migration of human primary monocytes towards inflammatory chemokines using a trans-well assay. For a broad analysis, several chemoattractants which serve as ligands for different chemokine receptors were used. Two assays were performed in triplicates for each chemoattractant.

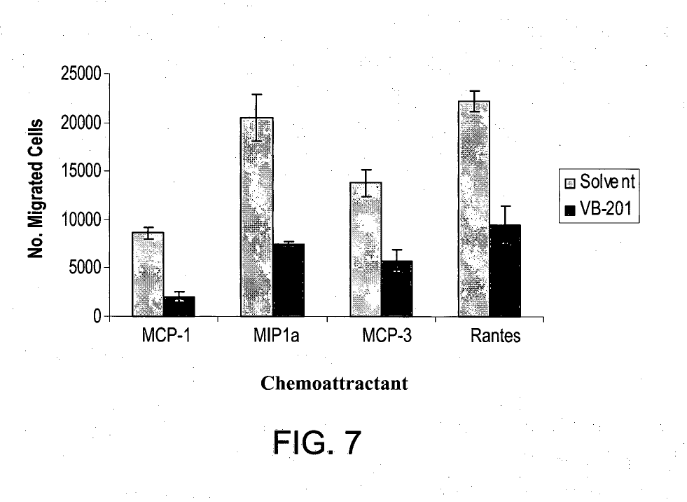

As shown in FIG. 7, VB-201 significantly (P<0.05) reduced migration of human primary monocytes towards all of the following monocytes attractants: MCP-1, MCP-3, MIP-1α and RANTES. Similar response was observed when human endothelial cell medium was used as an attractant.

The inhibition of monocyte migration exhibited by VB-201 in vitro was dose-dependent.

These results indicate that VB-201 can act as a significant inhibitor of immune cell migration.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1.-56. (canceled)

57. A method of treating or preventing an inflammatory disease or disorder associated with an endogenous oxidized lipid comprising orally administering to a human in need thereof a unit dosage form comprising VB-201 or a pharmaceutically acceptable salt thereof, wherein if administered to a human daily for 14 consecutive days, the unit dosage form has a pharmacokinetic profile of VB-201 in plasma having one or more characteristics selected from the group consisting of:
a) an AUC₍₀₋₂₄ₕ₎ of from 3,875 to 8,601 ng·h/mL, from 8,829 to 16,405 ng·h/mL, from 11,212 to 29,894 ng·h/mL, from 19,816 to 41,294 ng·h/mL, from 30,601 to 64,185 ng·h/mL or from 68,028 to 111,530 ng·h/mL; wherein the AUC₍₀₋₂₄ₕ₎ is measured on day 14;
b) a Cₘₐₓ of from 218.17 to 462.45 ng/mL, from 476.21 to 855.39 ng/mL, from 701.14 to 1,850.16 ng/mL, from 1,093.41 to 2,549.39 ng/mL, from 1,744.10 to 3,345.10 ng/mL or from 3,889.90 to 5,750.5 ng/mL; wherein the Cₘₐₓ of VB-201 is measured on day 14;
c) an AUC₍₀₋₂₄ₕ₎ of from 916 to 2,546 ng·h/mL, from 1,812 to 5,176 ng·h/mL, from 2,751 to 5,621 ng·h/mL, from 3,162 to 9,656 ng·h/mL or from 4,388 to 7,578 ng·h/mL, wherein the AUC₍₀₋₂₄ₕ₎ is measured on day 1; and
d) a Cₘₐₓ of from 79.72 to 154.96 ng/mL, from 147.56 to 308.28 ng/mL, from 183.50 to 404.96 ng/mL, from 213.76 to 619.40 ng/mL or from 636.30 to 1211.10 ng/mL; wherein the Cₘₐₓ of VB-201 is measured on day 1.

58. The method of claim 57, wherein if administered to a human daily for 14 consecutive days, the unit dosage form has a pharmacokinetic profile of VB-201 in plasma having a Cₘₐₓ of from 218.17 to 462.45 ng/mL, from 476.21 to 855.39 ng/mL, from 701.14 to 1,850.16 ng/mL, from 1,093.41 to 2,549.39 ng/mL, from 1,744.10 to 3,345.10 ng/mL or from 3,889.90 to 5,750.5 ng/mL; wherein the Cₘₐₓ of VB-201 is measured on day 14.

59. The method of claim 58, wherein if administered to a human daily for 14 consecutive days, the unit dosage form has a pharmacokinetic profile of VB-201 in plasma having a Tₘₐₓ of from 2.14 to 17.2 h, from 3.44 to 18.56 h, from 4.60 to 10.06 h, from 2.31 to 17.69 h, from 2.2 to 6.2 h or from 4.9 to 7.1 h, wherein the Tₘₐₓ of VB-201 is measured on day 14.

60. The method of claim 58, wherein if administered to a human daily for 14 consecutive days, the unit dosage form has a pharmacokinetic profile of VB-201 in plasma having an AUC₍₀₋₂₄ₕ₎ of from 3,875 to 8,601 ng·h/mL, from 8,829 to 16,405 ng·h/mL, from 11,212 to 29,894 ng·h/mL, from 19,816 to 41,294 ng·h/mL, from 30,601 to 64,185 ng·h/mL or from 68,028 to 111,530 ng·h/mL; wherein the AUC₍₀₋₂₄ₕ₎ is measured on day 14.

61. The method of claim 57, wherein if administered to a human daily for 14 consecutive days, the unit dosage form has a pharmacokinetic profile of VB-201 in plasma having a Cₘₐₓ of from 79.72 to 154.96 ng/mL, from 147.56 to 308.28 ng/mL, from 183.50 to 404.96 ng/mL, from 213.76 to 619.40 ng/mL or from 636.30 to 1211.10 ng/mL; wherein the Cₘₐₓ of VB-201 is measured on day 1.

62. The method of claim 61, wherein if administered to a human daily for 14 consecutive days, the unit dosage form has a pharmacokinetic profile of VB-201 in plasma having an AUC₍₀₋₂₄ₕ₎ of from 916 to 2,546 ng·h/mL, from 1,812 to 5,176 ng·h/mL, from 2,751 to 5,621 ng·h/mL, from 3,162 to 9,656 ng·h/mL or from 4,388 to 7,578 ng·h/mL, wherein the AUC₍₀₋₂₄ₕ₎ is measured on day 1.

63. The method of claim 57, wherein the unit dosage form comprises 5 mg, 10 mg, 20 mg, 30 mg, 40 mg or 80 mg VB-201.

64. The method of claim 57, wherein the unit dosage form is administered during a meal.

65. The method of claim 57, wherein the unit dosage form comprises 20 mg VB-201.

66. The method of claim 57, wherein the unit dosage form comprises 30 mg VB-20.

67. The method of claim 57, wherein the unit dosage form comprises 40 mg VB-201.

68. The method of claim 57, wherein the unit dosage form comprises 80 mg VB-201.

69. The method of claim 57, wherein the inflammatory disease or disorder is psoriasis.

70. The method of claim 57, wherein the inflammatory disease or disorder is selected from the group consisting of rheumatoid arthritis, multiple sclerosis, inflammatory bowel disease, atherosclerosis, and an inflammation of a carotid artery or inflammation of an aorta.

71. The method of claim 70, wherein the inflammatory disease or disorder is an inflammation of a carotid artery or inflammation of an aorta.

72. A unit dosage form comprising VB-201 or a pharmaceutically acceptable salt thereof, wherein if administered to a human daily for 14 consecutive days, the unit dosage form has a pharmacokinetic profile of VB-201 in plasma having one or more characteristics selected from the group consisting of:
a) an AUC₍₀₋₂₄ₕ₎ of from 3,875 to 8,601 ng·h/mL, from 8,829 to 16,405 ng·h/mL, from 11,212 to 29,894 ng·h/mL, from 19,816 to 41,294 ng·h/mL, from 30,601 to 64,185 ng·h/mL, or from 68,028 to 111,530 ng·h/mL; wherein the AUC₍₀₋₂₄ₕ₎ is measured on day 14;
b) a Cₘₐₓ of from 218.17 to 462.45 ng/mL, from 476.21 to 855.39 ng/mL, from 701.14 to 1,850.16 ng/mL, from 1,093.41 to 2,549.39 ng/mL, from 1,744.10 to 3,345.10 ng/mL, or from 3,889.90 to 5,750.5 ng/mL; wherein the Cₘₐₓ of VB-201 is measured on day 14;
an AUC₍₀₋₂₄ₕ₎ of from 916 to 2,546 ng·h/mL, from 1,812 to 5,176 ng·h/mL, from 2,751 to 5,621 ng·h/mL, from 3,162 to 9,656 ng·h/mL, or from 4,388 to 7,578 ng·h/mL, wherein the AUC₍₀₋₂₄ₕ₎ is measured on day 1; and
d) a Cₘₐₓ of from 79.72 to 154.96 ng/mL, from 147.56 to 308.28 ng/mL, from 183.50 to 404.96 ng/mL, from 213.76 to 619.40 ng/mL or from 636.30 to 1211.10 ng/mL; wherein the Cₘₐₓ of VB-201 is measured on day 1.

73. The unit dosage form of claim 72, wherein the unit dosage form comprises 80 mg VB-201, wherein if administered to a human daily for 14 consecutive days, the unit dosage form has a pharmacokinetic profile of VB-201 in plasma having
a) an AUC₍₀₋₂₄ₕ₎ of from 68,028 to 111,530 ng·h/mL; or
b) a Cₘₐₓ of from 3,889.90 to 5,750.5 ng/mL;
wherein the AUC₍₀₋₂₄ₕ₎ or Cₘₐₓ of VB-201 is measured on day 14.

74. The unit dosage form of claim 72, wherein the unit dosage form comprises 40 mg VB-201, wherein if administered to a human daily for 14 consecutive days, the unit dosage form has a pharmacokinetic profile of VB-201 in plasma haying
a) an AUC₍₀₋₂₄ₕ₎ of from 30,601 to 64,185 ng·h/mL, wherein the AUC₍₀₋₂₄ₕ₎ of VB-201 is measured on day 14;
b) a Cₘₐₓ of from 1,744.10 to 3,345.10 ng/mL, wherein the Cₘₐₓ of VB-201 is measured on day 14;
c) an AUC₍₀₋₂₄ₕ₎ of from 4,388 to 7,578 ng·h/mL, wherein the AUC₍₀₋₂₄ₕ₎ of VB-201 is measured on day 1; or
d) a Cₘₐₓ of from 636.30 to 1211.10 ng/mL, wherein the Cₘₐₓ of VB-201 is measured on day 1.

75. A method of treating or preventing an inflammatory disease or disorder associated with an endogenous oxidized lipid comprising administering to a patient in need thereof the unit dosage form of claim 73 or claim 74.

76. The method of claim 75, wherein the inflammatory disease or disorder is psoriasis.

77. The method of claim 75, wherein the inflammatory disease or disorder is selected from the group consisting of rheumatoid arthritis, multiple sclerosis, inflammatory bowel disease, atherosclerosis, and an inflammation of a carotid artery or inflammation of an aorta.

78. The method of claim 77, wherein the inflammatory disease or disorder is an inflammation of a carotid artery or inflammation of an aorta.
